(19) United States
(12) Patent Application Publication
(10) Pub. No.: US 2025/0083117 A1
(43) Pub. Date: Mar. 13, 2025

(54) MICROARRAYS AND METHODS

(71) Applicant: Verndari, Inc., Sacramento, CA (US)

(72) Inventor: Daniel R. Henderson, Sacramento, CA (US)

(21) Appl. No.: 18/665,335

(22) Filed: May 15, 2024

Related U.S. Application Data
(63) Continuation of application No. 16/323,234, filed on Feb. 4, 2019, now abandoned.
(60) Provisional application No. 62/508,861, filed on May 19, 2017.
(60) Provisional application No. 62/460,574, filed on Feb. 17, 2017.
(60) Provisional application No. 62/370,416, filed on Aug. 3, 2016.

(51) Int. Cl. B01J 19/00; A61K 9/00; A61K 47/26; A61K 47/38; A61M 37/00; A61P 31/12; A61P 31/16; B01L 3/00

(52) U.S. Cl. CPC B01J 19/0046; A61K 9/0021; A61K 47/26; A61K 47/38; A61M 37/0015; A61P 31/12; A61P 31/16; B01L 3/502761; A61M 2037/0038; A61M 2037/0053; B01J 2219/00432; B01J 2219/00605; B01J 2219/00659; B01L 2200/10

(57) ABSTRACT

The present disclosure describes microtips, MicroArrays, MicroArray Patches comprising MicroArrays, delivery devices, and methods of manufacturing and methods of using same. In some embodiments, a substance-loaded MicroArray is prepared by photochemically etching MicroArray outlines in a substrate sheet, the MicroArrays comprising a plurality of microtips, configuring reservoirs in each microtip such as by photochemical half etching, filling each reservoir with a substance to be delivered, and then bending each of the microtips out of planarity such that each microtip comprises a substance-loaded projection disposed an angle relative to the generally planar substrate sheet.

### CROSS-REFERENCE

This application is a continuation of U.S. application Ser. No. 16/323,234, filed Feb. 4, 2019; which is a 371 of International Application No. PCT/US2017/045161, filed Aug. 2, 2017, which claims the benefit of U.S. Application Ser. No. 62/370,416 filed Aug. 3, 2016; U.S. Application Ser. No. 62/460,574 filed Feb. 17, 2017; and U.S. Application Ser. No. 62/508,861 filed May 19, 2017, which are hereby incorporated by reference in their entirety.

### SUMMARY OF THE INVENTION

The present disclosure relates generally to medical devices comprising microtips and in particular to microtips, MicroArrays, MicroArray Patches comprising MicroArrays, kits comprising MicroArrays and packaging, dispensing devices for delivering microtip systems, and methods of manufacturing and methods for using same.

Disclosed herein, in certain embodiments, are MicroArrays comprising: a substantially planar substrate further comprising a plurality of substance-loaded microtip projections, each of said microtip projections projecting at an angle relative to the substantially planar substrate, wherein each of said microtip projections is hingeably attached to said substrate. In some embodiments, said angle is from about 50° to about 90° relative to said substantially planar substrate. In some embodiments, said microtip projections each further comprise a depression, and wherein said substance is loaded in said depressions. In some embodiments, said plurality of microtip projections form a grid pattern having a microtip density of about 25 microtip projections per square centimeter of substrate surface area. In some embodiments, said substantially planar substrate comprises a 25 micron to 150 micron thick metal sheet. In some embodiments, said metal is chosen from the group consisting of titanium, stainless steel, nickel, and mixtures thereof. In some embodiments, said substantially planar substrate comprises a plastic sheet of about 0.5 micron to 200 micron thickness. In some embodiments, said plastic is a thermoplastic material.

Disclosed herein, in certain embodiment, are MicroArrays comprising: a substantially planar substrate further comprising a plurality of substance-loaded microtip projections, each of said microtip projections projecting at an angle relative to the substantially planar substrate, said array formed by the process comprising: (a) providing the substrate; (b) etching a plurality of microtips in said substrate; (c) configuring a reservoir into each microtip; (d) loading an amount of a substance into each reservoir; and (c) bending each microtip out of planarity to an angle relative to the plane of the substrate to create each microtip projection. In some embodiments, said angle is from about 50° to about 90° relative to said substrate. In some embodiments, the step of configuring a reservoir comprises the etching of each microtip in a photochemical etching operation. In some embodiments, the step of etching a plurality of microtips and the step of configuring a reservoir into each microtip occur simultaneously. In some embodiments, the step of configuring a reservoir into each microtip comprises denting the substrate with the appropriate shaped tool. In some embodiments, the step of configuring a reservoir into each microtip comprises laser ablation of substrate material thickness. In some embodiments, said plurality of microtips comprises a microtip density of about 25 microtips per square centimeter of substrate. In some embodiments, said substrate comprises 25 to 150 micron thick metal sheet material. In some embodiments, said substrate comprises 0.5 to 200 micron thick plastic sheet material. In some embodiments, each microtip of said plurality of microtips further comprises (a) a hingeable portion at each proximal end of each microtip, attaching said microtips to said substrate; and (b) a beveled edge.

Disclosed herein, in certain embodiments, are methods of manufacturing a MicroArray comprising: (a) providing a substrate; (b) cutting a plurality of MicroArray outlines in the substrate, each MicroArray comprising a plurality of microtips; (c) configuring a reservoir into each microtip of said plurality of microtips; (d) dispensing an amount of a substance into each reservoir; and (e) bending each microtip of said plurality of microtips out of planarity to an angle relative to the plane of the substrate; and (f) excising individual MicroArrays from the substrate. In some embodiments, said angle is from about 50° to about 90° relative to said substrate. In some embodiments, the step of cutting a plurality of MicroArrays into the substrate comprises photochemical etching of the substrate. In some embodiments, the step of configuring a reservoir into each microtip comprises the photochemical etching of a portion of the thickness of the substrate at each microtip. In some embodiments, the steps of cutting a plurality of MicroArrays into the substrate and configuring a reservoir into each microtip comprise simultaneous photochemical etching processes. In some embodiments, the step of configuring a reservoir into each microtip comprises denting each microtip with a punch. In some embodiments, the step of cutting a plurality of microtips comprises die-cutting the substrate with the appropriate shaped tool. In some embodiments, the step of cutting a plurality of microtips comprises laser ablation. In some embodiments, the step of configuring a reservoir into each microtip comprises laser ablation of a portion of the thickness of the substrate at each microtip. In some embodiments, said plurality of microtips comprises a microtip density of about 25 microtips per square centimeter of substrate. In some embodiments, said amount of step (d) comprises from about 0.1 nL to about 5 nL of said substance. In some embodiments, said amount of step (d) comprises from about 0.2 ng to about 5 μg of said substance. In some embodiments, each microtip of said plurality of microtips comprises both a sharp distal end and a hingeable portion at a proximal end, said hingeable portion attaching each microtip to said substrate. In some embodiments, said substance is selected from the group consisting of an API, a mixture of APIs, a pharmaceutical composition, a therapeutic material, a therapeutic composition, a homeopathic material, a homeopathic composition, a cosmetic preparation, a vaccine, a medicament, an herb, a solvent, and mixtures thereof. In some embodiments, the photochemical etching of a portion of the thickness of the substrate at each microtip comprises the removal of up to about 80% of the thickness of the substrate. In some embodiments, said photochemical etching of a portion of the thickness of the substrate at each microtip comprises photochemical half etching on one side of the substrate. In some embodiments, said microtips measure approximately 475 μm in length and approximately 200 μm in width. In some embodiments, said vaccine is a cancer vaccine. In some embodiments, said vaccine is effective against a virus, a bacterium, or a fungus. In some embodiments, the substrate comprises a plurality of MicroArray outlines arranged into a plurality of rows and a plurality of columns. In some embodiments, the substrate further comprises a plurality of fiducial markers. In some embodiments, the substrate comprises MicroArray outlines arranged in rows of at least 10 MicroArray outlines per row. In some embodiments, the substrate comprises MicroArray outlines arranged in columns of at least 10 MicroArray outlines per column. In some embodiments, the substrate comprises MicroArray outlines arranged in columns of at least 50 MicroArray outlines per column. In some embodiments, a microfluidic dispensing device dispenses the substance into the plurality of microtip reservoirs. In some embodiments, the microfluidic dispensing device is a multi-channel microfluidic dispensing device. In some embodiments, the multi-channel microfluidic dispensing device is operably linked to an imaging system. In some embodiments, the substrate comprises a plurality of MicroArray outlines arranged into a plurality of rows and a plurality of columns, wherein the substrate further comprises a plurality of fiducial markers, and wherein the imaging system utilizes the spatial organization of the fiducial markers to align a dispensing nozzle of the multi-channel microfluidic dispensing device over a row of MicroArrays. In some embodiments, the substance is formulated as a sugar glass. In some embodiments, the sugar glass comprises trehalose. In some embodiments, a forming press bends the plurality of microtips out of planarity to an angle relative to the plane of the substrate. In some embodiments, the forming press comprises a plurality of forming supports and a plurality of forming dies. In some embodiments, each forming die in the plurality of forming dies comprises a plurality of projections that bend the microtips out of planarity to an angle relative to the plane of the substrate. In some embodiments, each forming support in the plurality of forming supports comprises a plurality of microtip clearance areas that allows the individual microtips to bend out of planarity to an angle relative to the plane of the substrate. In some embodiments, the forming press presses the plurality of forming dies and the plurality of forming supports together, and wherein the plurality of projections in each forming die bend each microtip of the plurality of microtips out of planarity with the substrate and into the microtip clearance area of the forming support. In some embodiments, a punch press excises the individual MicroArrays from the substrate. In some embodiments, the punch press comprises a punch array comprising a plurality of punch dies and a clamp array comprising a plurality of clamps. In some embodiments, the substrate comprises a plurality of MicroArray outlines arranged into a plurality of rows and a plurality of columns, and wherein the punch press presses the punch array and the clamp array together to excise individual MicroArrays in a row of MicroArrays.

Disclosed herein, in certain embodiments, are MicroArrays comprising: a substantially planar substrate further comprising a plurality of substance-loaded microtips, each one of said microtips projecting at an angle relative to the substantially planar substrate further comprising a hinged portion, wherein each one of said microtips is hingeably attached to said substrate by the hinged region; and wherein each one of said microtips further comprises a beveled edge and a reservoir. In some embodiments, said angle is from about 50° to about 90° relative to said substantially planar substrate. In some embodiments, said substance is loaded in said reservoirs. In some embodiments, said plurality of microtips form a grid pattern having a microtip density of about 25 microtips per square centimeter of substrate surface area. In some embodiments, said substantially planar substrate comprises a 25 micron to 150 micron thick metal sheet. In some embodiments, said metal is chosen from the group consisting of titanium, stainless steel, nickel, and mixtures thereof. In some embodiments, said substantially planar substrate comprises a plastic sheet of about 0.5 micron to about 200 micron thickness. In some embodiments, said plastic is a thermoplastic material. In some embodiments, the beveled edge is a double beveled edge, a top beveled edge, a bottom beveled edge, a double concave beveled edge, a top concave beveled edge, a bottom concave beveled edge, or a concave beveled edge. In some embodiments, the microtips have a length of about 600 to about 800 microns. In some embodiments, the microtips have a width of about 50 to about 350 microns. In some embodiments, the reservoirs have a depth of about 20 to about 50 microns. In some embodiments, the MicroArrays further comprise a “pick-and-place” point. In some embodiments, the reservoir is an enclosed reservoir or an open reservoir.

Disclosed herein, in certain embodiments, are methods of manufacturing a MicroArray comprising: (a) providing a substrate; (b) cutting a plurality of microtip outlines in the substrate to create a plurality of microtips in each MicroArray; (c) configuring a reservoir into each microtip of said plurality of microtips; (d) dispensing an amount of a substance into each reservoir; (e) bending each microtip of said plurality of microtips out of planarity to an angle relative to the plane of the substrate; and (f) excising the MicroArray from the substrate. In some embodiments, said angle is from about 45° to about 135° relative to said substrate. In some embodiments, the step of cutting a plurality of microtip outlines into the substrate comprises photochemical etching of the substrate. In some embodiments, the step of configuring a reservoir into each microtip comprises the photochemical etching of a portion of the thickness of the substrate at each microtip. In some embodiments, the steps of cutting a plurality of microtip outlines into the substrate and configuring a reservoir into each microtip comprise simultaneous photochemical etching processes. In some embodiments, the step of configuring a reservoir into each microtip comprises denting each microtip with a punch. In some embodiments, the methods comprise a simultaneous photochemical etching the step of cutting a plurality of microtip outlines comprises die-cutting the substrate with the appropriate shaped tool. In some embodiments, the methods comprise a simultaneous photochemical etching the step of cutting a plurality of microtip outlines comprises laser ablation. In some embodiments, the methods comprise a simultaneous photochemical etching the step of configuring a reservoir into each microtip comprises laser ablation of a portion of the thickness of the substrate at each microtip. In some embodiments, the methods comprise a simultaneous photochemical etching said plurality of microtips comprises a microtip density of about 25 microtips per square centimeter of substrate. In some embodiments, said amount of step (d) comprises from about 0.1 nL to about 5 nL of said substance. In some embodiments, said amount of step (d) comprises from about 0.2 ng to about 5 μg of said substance. In some embodiments, each microtip of said plurality of microtips comprises both a sharp distal end and a hinged portion at a proximal end, said hinged portion attaching each microtip to said substrate. In some embodiments, said substance is selected from the group consisting of an API, a mixture of APIs, a pharmaceutical composition, a therapeutic material, a therapeutic composition, a homeopathic material, a homeopathic composition, a cosmetic preparation, a vaccine, a medicament, an herb, a solvent, and mixtures thereof. In some embodiments, the photochemical etching of a portion of the thickness of the substrate at each microtip comprises the removal of up to about 80% of the thickness of the substrate. In some embodiments, said photochemical etching of a portion of the thickness of the substrate at each microtip comprises photochemical half etching on one side of the substrate. In some embodiments, said microtips measure approximately 475 μm in length and approximately 200 μm in width. In some embodiments, said vaccine is a cancer vaccine. In some embodiments, said vaccine is effective against a virus, a bacterium, or a fungus. In some embodiments, the substrate comprises a plurality of MicroArray outlines arranged into a plurality of rows and a plurality of columns. In some embodiments, the substrate further comprises a plurality of fiducial markers. In some embodiments, the substrate comprises MicroArray outlines arranged in rows of at least 10 MicroArray outlines per row. In some embodiments, the substrate comprises MicroArray outlines arranged in columns of at least 10 MicroArray outlines per column. In some embodiments, the substrate comprises MicroArray outlines arranged in columns of at least 50 MicroArray outlines per column. In some embodiments, a microfluidic dispensing device dispenses the substance into the plurality of microtip reservoirs. In some embodiments, the microfluidic dispensing device is a multi-channel microfluidic dispensing device. In some embodiments, the multi-channel microfluidic dispensing device is operably linked to an SMT system. In some embodiments, the substrate comprises a plurality of MicroArray outlines arranged into a plurality of rows and a plurality of columns, wherein the substrate further comprises a plurality of fiducial markers, and wherein the imaging system utilizes the spatial organization of the fiducial markers to align a dispensing nozzle of the multi-channel microfluidic dispensing device over a row of MicroArrays. In some embodiments, the substance is formulated as a sugar glass. In some embodiments, the sugar glass comprises trehalose. In some embodiments, a forming press bends the plurality of microtips out of planarity to an angle relative to the plane of the substrate. In some embodiments, the forming press comprises a plurality of forming supports and a plurality of forming dies. In some embodiments, each forming die in the plurality of forming dies comprises a plurality of projections that bend the microtips out of planarity to an angle relative to the plane of the substrate. In some embodiments, each forming support in the plurality of forming supports comprises a plurality of microtip clearance areas that allows the individual microtips to bend out of planarity to an angle relative to the plane of the substrate. In some embodiments, the forming press presses the plurality of forming dies and the plurality of forming supports together, and wherein the plurality of projections in each forming die bend each microtip of the plurality of microtips out of planarity with the substrate and into the microtip clearance area of the forming support. In some embodiments, a punch press excises the individual MicroArrays from the substrate. In some embodiments, the punch press comprises a punch array comprising a plurality of punch dies and a clamp array comprising a plurality of clamps. In some embodiments, the substrate comprises a plurality of MicroArray outlines arranged into a plurality of rows and a plurality of columns, and wherein the punch press presses the punch array and the clamp array together to excise individual MicroArrays in a row of MicroArrays.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 illustrates an embodiment of a microtip comprising an open reservoir 125A in accordance with the present disclosure.

FIG. 2 illustrates another embodiment of a microtip comprising an enclosed reservoir 125B and a straight edge 210 in accordance with the present disclosure.

FIG. 3 shows a close up image of a single microtip with an enclosed reservoir 125B; surface roughness is readily apparent. Also shown is a region of interest (ROI) 620, which is used by Surface Imaging and Metrology Software Leica Map to determine surface roughness.

FIGS. 4A-I illustrate the different types of finished edges of a microtip. FIG. 4A illustrates a microtip with a straight edge 210 displaying a dotted line intersecting the microtip longitudinally at its center. FIG. 4B shows a cross-sectional view of a microtip comprising a double beveled edge 330. FIG. 4C shows a cross-sectional view of a microtip comprising a top beveled edge 340. FIG. 4D shows a cross-sectional view of a microtip comprising a bottom beveled edge 350. FIG. 4E shows a cross-sectional view of a microtip comprising a straight edge 210 (i.e. a non-beveled edge). FIG. 4F shows a cross-sectional view of a microtip comprising a double concave beveled edge 360. FIG. 4G shows a cross-sectional view of a microtip comprising a top concave beveled edge 370. FIG. 4H shows a cross-sectional view of a microtip comprising a bottom concave beveled edge 380. FIG. 4I shows a cross-sectional view of a microtip comprising a concave beveled edge 390.

FIG. 5 illustrates an embodiment of a MicroArray depicting an individual MicroArray excised from a substrate sheet and containing flat microtips (i.e., in the X/Y plane) with empty reservoirs (i.e., unloaded with a substance) in accordance with the present disclosure, and further illustrates detail of same in a magnified portion.

FIG. 6 illustrates the filling of a substance 155 with a nozzle 150 into an enclosed reservoir 125B of a microtip in accordance with the present disclosure.

FIG. 7 illustrates an excised, filled MicroArray 174 in accordance with the present disclosure, wherein each microtip comprises a filled reservoir 126 and is projecting from the surface of the substrate 110 at an angle 400 into the Z-plane.

FIG. 8 illustrates an embodiment of a MicroArray patch 180 in accordance with the present disclosure, comprising an adhesive disc 160.

FIGS. 9A-B show an exemplary depiction of a 1 cm² 5×5 excised, empty, flat MicroArray 170 demonstrating a central vacuum “pick-up point” 220 for robotic automated “pick and place” systems. FIG. 9A shows a MicroArray with sharp corners 630. FIG. 9B shows a MicroArray with optional rounded corners 640.

FIG. 10 is an exemplary illustration of a 10×50 MicroArray sheet 240.

FIGS. 11A-B are illustrative cross-sections of an exemplary forming-die and upper forming support press demonstrating the production of Z-plane bent microtips. FIG. 11A shows a forming die 310 aligned directly beneath microtips extending from a MicroArray sheet 240. FIG. 11B shows bent microtips after the forming die 310 is pressed in an upward motion, as shown by the arrow.

FIG. 12 is an exemplary depiction of a production flow chart that results in the manufacture of sterile, individual MicroArrays with Z-plane bent sample-loaded microtips.

FIG. 13 is an exemplary depiction of a manufacturing flow layout that results in the production of sterile, packaged MicroArray patches.

FIG. 14 shows an exemplary image of sugar-glass influenza HA vaccine 480 loaded onto a microtip 126. 1% Congo Red was added to the vaccine formulation as a visualization aid. The microtip on the left demonstrates a vaccine-sugar glass after 48 hours of drying time at room temperature while the microtip on the right demonstrates a solid, dried, and intact vaccine-sugar glass after probing with a dissection needle.

FIG. 15 shows representative results from the application of a sugar-glass formulated MicroArray to pig skin samples. 600A-C are pig skin samples 1 minute, 5 minutes, and 20 minutes after application of a sugar-glass formulated MicroArray and before any rinsing with phosphate buffered saline (PBS). 600D-F are rinsed pig skin samples 1 minute, 5 minutes, and 20 minutes after the application of a sugar-glass formulated MicroArray and after subsequent rinsing of the pig skin sample with PBS.

FIG. 16 is a group of illustrations of exemplary MicroArray customized designs that are amenable to be utilized with the manufacturing methods disclosed herein.

FIGS. 17A-C show mouse titer values after a hepatitis B vaccine (Engerix-B) was administered either intramuscularly or via the MicroArray patches described herein. FIG. 17A shows titer values in mouse sera after an IM injection of with Engerix-B. FIG. 17B shows titer values in mouse sera after being injected with Engerix-B-loaded MicroArray patches. FIG. 17C compares the titer values in mouse sera after exposure to Engerix-B delivered via a MicroArray patch or intramuscularly.

FIG. 18 shows rat titer values after administration of an influenza vaccine, Fluarix. The influenza virus vaccine was administered to rats intradermally, intramuscularly, and via the MicroArray patches described herein.

### DETAILED DESCRIPTION OF THE INVENTION

Transdermal patches are known medical devices used for administrating substances such as drugs to patients in a convenient and non-invasive manner. However, transdermal patches require large chemical loading of an active pharmaceutical ingredient (“API”) or composition thereof on the patch, and often the inclusion of skin penetrants and other special ingredients in the composition to increase transdermal efficiency. Subcutaneous/intramuscular injection is a more efficient drug administration method, but, unlike a patch, is impractical for self-administration. Even healthcare workers must be specially trained on proper/safe injection techniques, and the amount of medical waste (syringes, needles, tourniquets, etc.) is substantial just for a single dose.

“Microtips” (also known as microneedles) provide an alternative to conventional transdermal patches and syringe injections. Microtips penetrate skin only to a depth of about 400 to 500 μm, an insufficient depth to reach nerves or blood vessels. Additionally, penetrating agents are not typically used with microtips, and thus microtips are not transdermal delivery devices per se. Microtips are able to penetrate the stratum corneum and the epidermis, but penetrate into only a portion of the dermis. Without full penetration of the dermis, microtips are more correctly referred to as “transepithelial delivery devices” rather than “transdermal delivery devices.” Microtips arranged in a small array (also known as a microneedle array, hereinafter referred to as a “MicroArray”), each coated with a very small amount of a drug or other substance, provide case of self-administration, reduced drug delivery costs, avoidance of hypodermic needle stick injuries, smaller dosage volumes, less fear compared to a home injection when self-administering, and reduced healthcare training burden. Microtips are painless because, as mentioned, the individual needles are too short in length to stimulate nerve endings.

Unfortunately, MicroArrays are complex, microscopic, engineering-intensive devices, and are, consequently, very difficult to precisely manufacture. Such complexity and precision is typically not amenable to rapid, high-volume manufacturing processes. Further, current methods for making drug-loaded microtips involve the inefficient and wasteful dipping or roll-coating of microtips with drug substances. For rare or difficult to manufacture drugs, such as polynucleotide vaccines for example, this wasteful drug application process is unacceptable, since it means certain vital drugs will not be available in sufficient supply in times of great need, such as during a global pandemic where literally hundreds of millions of drug doses are needed.

In various embodiments, the present disclosure provides microtips, MicroArrays, and MicroArray Patches comprising MicroArrays, kits comprising MicroArrays and packaging, dispensing devices for delivering microtip systems, and methods of manufacturing and methods for using same. In various aspects, the present disclosure provides new manufacturing processes useful for producing substance-loaded MicroArrays on a scale of tens of millions of arrays per week, without involving wasteful dipping or roll-coating steps. More specifically, the present disclosure provides the following:

In various aspects, the present disclosure provides a MicroArray comprising a substantially planar substrate further comprising a plurality of substance-loaded microtip projections, each of said microtip projections projecting at an angle relative to the substantially planar substrate, wherein each of said microtip projections is hingeably attached to said substrate. In some embodiments, the microtip projection angles range from about 45° to about 135° relative to the substantially planar substrate. In various examples, each microtip further comprises a depression in which the substance is loaded. In some embodiments, the MicroArray comprises a grid pattern having a microtip density of about 25 microtip projections per square centimeter of substrate surface area. In some embodiments, the substantially planar substrate comprises a 25 micron to 150 micron thick metal sheet, optionally referred to as a foil. In some embodiments, the metal is chosen from the group consisting of titanium, stainless steel, nickel, and mixtures thereof. In other embodiments, the substantially planar substrate comprises a plastic sheet of about 0.5 micron to 200 micron thickness, and the plastic sheet is a thermoplastic.

In other embodiments, the present disclosure provides a MicroArray comprising: a substantially planar substrate further comprising a plurality of substance-loaded microtip projections, each of said microtip projections projecting at an angle relative to the substantially planar substrate, said array formed by the process comprising: (i) providing the substrate; (ii) etching a plurality of microtips in said substrate; (iii) configuring a reservoir into each microtip; (iv) loading an amount of a substance into each reservoir; and (v) bending each microtip out of planarity to an angle relative to the plane of the substrate to create each microtip projection. In some embodiments, the microtips are bent at an angle from about 45° to about 135° relative to said substrate. In more specific embodiments, the step of configuring a reservoir comprises the etching of each microtip in a photochemical etching operation, and the step of etching a plurality of microtips and the step of configuring a reservoir into each microtip occurs simultaneously or stepwise in either order. In other examples, the step of configuring a reservoir into each microtip comprises denting the substrate with the appropriate shaped tool at each microtip location on the substrate. Alternatively, the step of configuring a reservoir into each microtip comprises photochemical etching of a portion of the substrate material thickness. In some embodiments, the step of configuring a reservoir into each microtip comprises laser ablation of substrate material thickness. In some embodiments, such MicroArrays have a microtip density of about 25 microtips per square centimeter of substrate, and the substrate is metal sheeting of 25 to 150 microns thickness. Alternatively, in some embodiments, the substrate is plastic rather than metal, and ranges from 0.5 to 200 microns thick. In some embodiments, this plastic substrate is a thermoplastic that is softened by heating and returned to a rigid state by cooling. In various embodiments, each microtip of the MicroArray further comprises a hingeable portion at each proximal end of each microtip, attaching said microtips to said substrate. The hingeable portion is useful for locally bending each microtip out of the plane of the substrate.

In various embodiments, the present disclosure also provides for a method of manufacturing a MicroArray comprising: (i) providing a substrate; (ii) cutting a plurality of microtips in said substrate; (iii) configuring a reservoir into each microtip of said plurality of microtips; (iv) dispensing an amount of a substance into each reservoir; and (v) bending each microtip of said plurality of microtips out of planarity to an angle relative to the plane of the substrate. In some embodiments, the angle at which each microtip projects from the substrate is from about 50° to about 90° relative to said substrate. In some embodiments, the angle at which each microtip projects from the substrate is about 90° relative to said substrate. In various examples, the step of cutting a plurality of microtips in the substrate comprises photochemical etching of the substrate, and the step of configuring a reservoir into each microtip comprises photochemical etching of a portion of the thickness of the substrate at each microtip. In some embodiments, the photochemical etching of the microtips and the photochemical etching of each reservoir on each microtip is conducted simultaneously, in one photochemical etching operation. Alternatively, the step of configuring a reservoir comprises denting each microtip in the substrate using a punch. Furthermore, in some embodiments, the step of cutting a plurality of microtips comprises die-cutting the substrate with the appropriate shaped tool. In other aspects, the step of cutting a plurality of microtips in the substrate comprises photochemical etching and/or laser ablation, and the photochemical etching and/or laser ablation is used to remove a portion of the thickness of the substrate at each microtip to fashion each reservoir. In various embodiments, the MicroArray has a microtip density of about 25 microtips per square centimeter of substrate. In some embodiments, the volume of substance loaded on each microtip ranges from about 0.1 nL to about 5 nL, from about 1 nL to about 2 nL, from about 2 nL to about 3 nL, from about 3 nL to about 4 nL, from about 4 nL to about 5 nL, from about 5 nL to about 6 nL, from about 6 nL to about 7 nL, from about 7 nL to about 8 nL, from about 8 nL to about 9 nL, from about 9 nL to about 10 nL, from about 10 nL to about 15 nL, from about 15 nL to about 20 nL, from about 20 nL to about 25 nL, from about 25 nL to about 30 nL, from about 30 nL to about 35 nL, or from about 35 nL to about 40 nL of said substance. In some embodiments, an aliquot volume of a substance is loaded onto each microtip of a MicroArray in multiple dispensations of the substance. For example, in some embodiments, an aliquot volume of a substance is loaded onto each microtip and allowed to dry before another aliquot volume of a substance is loaded onto each microtip. In some embodiments, for example, 10 nL of a substance is loaded onto each microtip of a MicroArray and allowed to dry, whereby another 10 nL of substance is loading onto the MicroArray for a total of 20 nL of total volume loaded onto the MicroArray. Any suitable number of successive iterations of the loading, drying, and reloading procedure described above is specifically contemplated for use in the devices and methods disclosed herein.

In some embodiments, substance loading on each microtip by weight is from about 0.2 ng to about 5 μg, from about 10 ng to about 20 ng, from about 20 ng to about 30 ng, from about 30 ng to about 40 ng, from about 40 ng to about 50 ng, from about 50 ng to about 60 ng, from about 60 ng to about 70 ng, from about 70 ng to about 80 ng, from about 80 ng to about 90 ng, from about 90 ng to about 100 ng, from about 100 ng to about 200 ng, from about 200 ng to about 300 ng, from about 300 ng to about 400 ng, from about 400 ng to about 500 ng, from about 500 ng to about 600 ng, from about 600 ng to about 700 ng, from about 700 ng to about 800 ng, from about 800 ng to about 900 ng, from about 900 ng to about 1000 ng, from about 1 μg to about 1.5 μg, from about 1.5 μg to about 2 μg, from about 2 μg to about 2.5 μg, from about 2.5 μg to about 3 μg, from about 3 μg to about 3.5 μg, from about 3.5 μg to about 4 μg, from about 4 μg to about 4.5 μg, from about 4.5 μg to about 5 μg, from about 5 μg to about 10 μg, from about 10 μg to about 15 μg, from about 15 μg to about 20 μg, from about 20 μg to about 30 μg, from about 30 μg to about 40 μg, from about 40 μg to about 50 μg, from about 50 μg to about 100 μg of said substance per microtip. In some embodiments, each microtip of said plurality of microtips comprises both a sharp distal end and a hingeable portion at a proximal end, said hingeable portion attaching each microtip to said substrate. In various embodiments, the substance loaded on each microtip is selected from the group consisting of an API, a mixture of APIs, a pharmaceutical composition, a therapeutic material, a therapeutic composition, a homeopathic material, a homeopathic composition, a cosmetic preparation, a vaccine, a medicament, an herb, a solvent, and mixtures thereof. In various aspects, a vaccine loaded on each microtip is effective against a disease caused by a virus, a bacterium, or a fungus. Further, and without limiting any of the foregoing, in some embodiments, the vaccine loaded on each microtip is effective against: cancer, influenza, chickenpox, smallpox, diphtheria, hepatitis A, hepatitis B, hepatitis E, haemophilus influenza type b (Hib), Japanese encephalitis, herpes zoster, human papilloma virus (HPV), viral, bacterial or fungal meningitis, meningococcal meningitis, amoebas infections, measles, mumps, polio, pneumonia, rabies, rotavirus, rubella, tetanus, tick-borne encephalitis, typhoid fever, yellow fever, campylobacter jejuni, Chagas disease, chikungunya, enterotoxigenic E-coli, enterovirus 71 (EV71), Group B streptococcus (GBS), HIV-1, human hookworm disease, leishmaniasis disease, nipah virus, nontyphoidal salmonella disease, respiratory syncytial virus (RSV), schistosomiasis disease, shigella, staphylococcus aureus, streptococcus pneumoniae, streptococcus pyrogenes, pertussis, any other childhood or adult disease caused by any type and/or species of any organism, Ebola, Zika, H1N1, swine flu, bird flu, malaria, cholera, dengue, anthrax, or tuberculosis.

### Definitions

As used herein, the term “microtip” refers to a substance delivery device capable of delivering a substance, such as a drug, to a patient. Conventional microtips typically have shapes similar to the tip of a beveled hypodermic needle (e.g. lancet, trocar, vet point, etc.) or other odd shapes (conical, tubular, etc.), albeit much smaller than typical needles, and comprise at least one bore, channel, port, tubular chamber, reservoir, or other structural feature or combinations thereof, such that a drug or other substance is disposed on (or inside) the microtips for subsequent administration to a patient. In one set of examples, a microtip is a microscopically dimensioned, flat, arrowhead-or spear-shaped piece of metal or plastic, onto which a dose of a substance is coated and optionally dried. In some aspects, microtips are engineered to partially or fully dissolve when in place in the patient. Microtips in general, and in accordance to the present invention, are quite small, (i.e. “micron scale”). More precise dimensions for microtips in accordance to the present disclosure are discussed herein.

As used herein, the term “MicroArray” refers to a substance delivery system comprising more than one microtip, such as a plurality of microtips (dozens, hundreds, even thousands), disposed on a relatively planar, “sheet-like” substrate. In various embodiments, a plurality of microtips in a MicroArray is arranged in a particular pattern. As a non-limiting example, a MicroArray in accordance to the present disclose consists of 25 individual microtips, uniformly spaced in a 5×5 grid pattern on a square flat substrate measuring 0.25 cm², with the point of each microtip projecting orthogonally (i.e. about 90°) from the planar surface of the substrate. Macroscopically, such an array appears two-dimensional. However, upon closer scrutiny, such as by examination under magnification, a MicroArray is seen to really be three-dimensional. That is, in some embodiments, a MicroArray comprises a substantially planar, two-dimensional sheet-like substrate with the microtips projecting out into the Z-plane from the substrate surface at an angle of about 90° relative to the planar substrate surface.

As used herein, the “x/y-plane” or “x/y-directions” refers to the surface of a relatively flat, planar, shect-like substrate. The term “z-direction” refers to the direction 90° relative to the x/y-plane. The x, y, and z-axis are the same as in basic geometry, although for purposes of describing the present invention, the sheet-like substrate of a MicroArray (or a transdermal patch comprising a MicroArray) is oriented with its larger dimensions in the x/y plane and its thickness (generally very thin) along the z-axis. When referring to a MicroArray herein, the microtips are oriented in the z-direction, and the planar sheet-like substrate, from which the microtips project, is oriented in the x/y-plane.

As used herein, the “distal end” of a microtip refers to the pointed (beveled, trocar, sharpened, conical, etc.) end of a microtip, configured for piercing the skin of a patient when the microtip is brought into contact with the individual. Consequently, the “proximal end” of a microtip refers to the blunt end, or the end opposite the sharp end, which will generally be anchored to the substrate. Thus, in a MicroArray, the distal ends of the microtips are at a measureable distance from the planar surface of the MicroArray substrate, whereas the proximal ends of the microtips are generally attached to the substrate and are, at least in some instances, contiguous with the substrate and comprising the same material as the substrate. In some embodiments, the substance to be delivered by the microtip is disposed on all or any portion of the microtip, such as at the distal or proximal ends, or on or in any portion between the extreme distal and proximal ends.

As used herein, the term “substrate” refers to the relatively thin, sheet-like portion of a MicroArray, used to support the microtips in a particular directional orientation, and in some instances, used as the material of construction for the microtips. “Sheet-like” means that the dimensions of the substrate in the x-and y-directions measure many times greater than the thickness of the substrate in the z-direction. In various embodiments, a precursor substrate sheet in manufacturing measures many feet wide and long yet only microns or millimeters in thickness, and is used as the precursor for thousands of individual MicroArrays cut from the precursor substrate. In various embodiments, a MicroArray comprises a substrate having photochemically etched, laser-cut, die-cut, or electrochemically etched, and optionally electropolished, projections acting as microtips.

As used herein, the term “substance” refers to the material to be disposed on a MicroArray and made available for delivery to a patient when the MicroArray is affixed to the skin of the patient. “Substance” herein is intended to have a very broad scope, and includes such things as active drugs (e.g. one molecular substance), combinations of drugs, pharmaceutical compositions, therapeutic materials and compositions thereof, homeopathic materials and compositions thereof, cosmetic preparations, vaccines, medicaments, herbs, solvents (e.g. DMSO), and the like. A “substance” for purposes herein comprises any physical form, such as for example, homogeneous liquid, emulsion, suspension, crystalline solid, amorphous solid, a coating of any viscosity, a hardened or polymerized coating, a previously liquid material subsequently dried, and the like. In various embodiments, a substance disposed on a MicroArray comprises a pure API (e.g. taxol). In other embodiments, a MicroArray is loaded with a vaccine based on non-virulent viruses, peptides rich in arginine, (e.g. oligoarginine, e.g. Arg8, or derivatives of the TaT protein to assist attachment to cells and invaginations into endosomes). In some embodiments, peptides functioning to aid release from endosomes are incorporated. For example, the processes herein allow application of very small amounts of materials such as vaccines, peptides, spermidine, various dendrimers, and/or polynucleotide stabilizing agents such as RNA stable onto microtips. In various embodiments, polynucleotide stabilizing agents on microtips facilitate increased shelf-life of API-loaded microtips. As discussed herein below, loaded microtips are further stabilized by special packaging.

In various embodiments, the term “substance” refers to a vaccine effective against at least one of, but not limited to: cancer, influenza, chickenpox, smallpox, diphtheria, hepatitis A, hepatitis B, hepatitis E, haemophilus influenza type b (Hib), Japanese encephalitis, herpes zoster, human papilloma virus (HPV), viral, bacterial or fungal meningitis, meningococcal meningitis, amoebas infections, measles, mumps, polio, pneumonia, rabies, rotavirus, rubella, tetanus, tick-borne encephalitis, typhoid fever, yellow fever, campylobacter jejuni, Chagas disease, chikungunya, enterotoxigenic E-coli, enterovirus 71 (EV71), Group B streptococcus (GBS), HIV-1, human hookworm disease, leishmaniasis disease, nipah virus, nontyphoidal salmonella disease, respiratory syncytial virus (RSV), schistosomiasis disease, shigella, staphylococcus aureus, streptococcus pneumoniae, streptococcus pyrogenes, pertussis, any other childhood or adult disease caused by any type and/or any species of any organism, Ebola, Zika, H1N1, swine flu, bird flu, malaria, cholera, dengue, anthrax, and tuberculosis.

In some embodiments, one or more substances are loaded onto microtips, such as to produce drug combinations.

The term “about” or “approximately” means within an acceptable error range for the particular value as determined by one of ordinary skill in the art, which will depend in part on how the value is measured or determined, e.g., the limitations of the measurement system. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 20.0degrees, 5.0 degrees, 10.0 degrees, 9.0 degrees, 8.0 degrees, 7.0 degrees, 6.0 degrees, 5.0degrees, 4.0 degrees, 3.0 degrees, 2.0 degrees, 1.0 degrees, 0.9 degrees, 0.8 degrees, 0.7 degrees, 0.6 degrees, 0.5 degrees, 0.4 degrees, 0.3 degrees, 0.2 degrees, 0.1 degrees, 0.09 degrees. 0.08 degrees, 0.07 degrees, 0.06 degrees, 0.05 degrees, 0.04 degrees, 0.03 degrees, 0.02 degrees or 0.01 degrees of a given value or range.

The terms “individual,” “patient,” or “subject” are used interchangeably. None of the terms require or are limited to situation characterized by the supervision (e.g., constant or intermittent) of a health care worker (e.g., a doctor, a registered nurse, a nurse practitioner, a physician's assistant, an orderly, or a hospice worker).

### Substrate Material

In various embodiments of the present disclosure, sheet-like substrate material for MicroArrays consist of a metal or a plastic, provided in sheet form and having a thickness of from about 0.5 micron to about 200 microns. In some instances, the sheet-like substrate material is from about 25 microns to about 150 microns thick, and in specific instances, about 75 microns thick. Non-limiting examples of substrates include: titanium, aluminum, stainless steel, nickel, copper, ruthenium, rhodium, palladium, silver, platinum, gold, polyethylene, polypropylene, polyethylene terephthalate, polycarbonate, and Teflon sheets, or composites thereof, with thicknesses ranging from about 0.5 micron to about 200 microns and length and width of any size deemed practical for further processing (e.g. up to dozens of feet wide and hundreds of feet long). In some embodiments, metal foil is commercially obtained, and is provided on spools of 2 to 3 feet wide by hundreds of feet long for use with the devices and methods disclosed herein. In some embodiments, these commercially available spools are loaded directly onto machines capable of photochemical etching of the foil.

In some embodiments, the sheet-like substrate comprises a single material or mixtures of materials, such as for example, mixtures of metals in a particular metal alloy or multi-layered sheets. In a representative example, a substrate comprises a 25 micron thick titanium sheet, such as the titanium sheets sold under the designation “Grade 2” from Hamilton Precision Metals, Lancaster, PA. In other examples, 304 stainless steel sheets or 316 stainless steel sheets are employed. Compared to titanium, stainless steel provides reduced cost and a more streamlined FDA approval process since 304 and 316 stainless steel is used for most hypodermic needles. Commercially available substrate material is ordinary provided in incremental “mil thicknesses,” such as for example, 1 mil, 2 mils, 3 mils, 4 mils, etc., (1 mil=25 microns). Thus, in some examples, 1 mil thick sheet material is used, (i.e. about 25 micron thick) for microtip production herein. In some embodiments, 2 mil thick sheet material is used (i.e. about 50 microns), for microtip production herein. In other embodiments, 3 mil thick sheet material is used (i.e. about 75 microns), for microtip production herein. In some embodiments, 4 mil thick sheet material is used (i.e. about 100 microns), for microtip production herein.

In some embodiments, for individual MicroArrays, the substrate x/y-dimensions will be much smaller than what's described above, such that practically sized medical patches are created. Thus, large precursor substrate sheets as described above will eventually be cut into much smaller individual MicroArrays, such as having a square, round, oval, triangular, or rectangular shape, or any other suitable shape, having a surface area of about 0.1 cm² to about 4 cm². For example, in some embodiments, MicroArrays in accordance to the present disclosure comprise a square substrate measuring about 0.5 cm×0.5 cm (and thus having a surface area of 0.25 cm²) up to about 2 cm×2 cm (thus having a surface area of 4 cm²), each with a thickness of about 0.5 to about 200 microns, preferably about 25 to about 75 microns. In some embodiments, MicroArrays in accordance to the present disclosure are about 1 cm², each with a thickness of about 0.5 to about 200 microns, preferably about 25 to about 75 microns. Projecting from each of these individual, small square substrates are as few as one individual microtip, or dozens, or up to hundreds of microtips.

In various embodiments, a sheet of substrate is photochemically etched, electrochemically etched, die-stamped, or laser-cut such that the microtips, once bent to project approximately orthogonally from the sheet, appear as projections emanating from the substrate sheet. This inventive aspect is discussed below.

### Microtips

In some embodiments, microtips in accordance with the present disclosure are configured into any suitable shape. For example, microtips are generally flat (i.e. substantially two-dimensional) or have some sort of three-dimensional shape (e.g. curved, indented, tubular, conical, etc.). In some embodiments, microtips are, for example, arrow-shaped, spear-shaped, lancet-shaped, or trocar-shaped, and are be flat, partially curved (such as to form a channel), tubular, conical, or comprise a recessed portion or indentation that acts as a reservoir for a substance.

In some embodiments, a microtip has a length (measured between distal and proximal end) of about 10-1000 microns and width of about 10-1000 microns. In some embodiments, a microtip has a length of about 400 to about 500 microns and a width of about 175 to about 250 microns. The thickness of a microtip corresponds to the thickness of the sheet of substrate material from which it is etched or cut, such as from about 0.5 to about 200 microns thick. In some embodiments, the thickness of the sheet of substrate is about 25 to about 75 microns. In other aspects, microtips are thicker than the sheet material used to form them, e.g. if the microtips are subsequently rolled into a curved shape or dented-in with an indentation to create three-dimensionality, or thinner than the sheet material, e.g. if material thickness is ablated away. In various embodiments, microtips within a MicroArray are generally arrow-shaped, measuring about 100 microns in height (i.e. the distance between distal and proximal ends), about 115 microns in width, and about 25 microns in thickness (each microtip having a surface area of about 5.75×10⁻⁵ cm²). In some examples, microtips range from about 175 microns to about 250 microns in width. In other variations, microtips measure up to about 750 microns in length. In some examples, microtips range from about 400 to about 500 microns in length.

In some embodiments, regardless of overall shape, each microtip further comprises at least one small recess, ablated region, indentation, or reservoir, appropriately dimensioned for use as a substance delivery reservoir. In some embodiments, an indentation is disposed at the distal end such as to include the distal edge of the microtip as the distal boundary of the indentation, or alternatively, an indentation is disposed at any position on the microtip. In various embodiments, a reservoir on a microtip (e.g. dented-in or recessed portion, or an ablated region) has a volume of about 0.1 nL to about 1 μL. In various examples, the volume of a reservoir on an individual microtip is from about 0.1 nL to about 5 nL, from about 1 nL to about 2 nL, from about 2 nL to about 3 nL, from about 3 nL to about 4 nL, from about 4 nL to about 5 nL, from about 5 nL to about 6 nL, from about 6 nL to about 7 nL, from about 7 nL to about 8 nL, from about 8 nL to about 9 nL, from about 9 nL to about 10 nL, from about 10 nL to about 15 nL, from about 15 nL to about 20 nL, from about 20 nL to about 25 nL, from about 25 nL to about 30 nL, from about 30 nL to about 35 nL, or from about 35 nL to about 40 nL. However, the range for reservoir volume is dependent on substrate thickness, with a thicker substrate allowing for deeper and hence larger volume reservoirs, regardless if the reservoir is dented in/extruded or is the result of etching and/or ablation of some of the thickness of the substrate. Also the length and width of each microtip dictates the available surface area for each reservoir. In photochemical half etching, for example, about 0-80% of the thickness of the metal foil substrate is removed to form reservoirs. Thus, for example, a 4 mil 304 stainless steel foil having thickness of 100 μm is photochemically half etched to about 50% to create a reservoir of 0-80 μm in depth. In other examples, 3 mil 304 stainless (75 μm thick) is photochemically half etched to about 50% of its thickness to provide for reservoirs having volumes of from about 1 nL to about 2 nL.

As mentioned, in some embodiments, microtips are metal or plastic, and are etched or otherwise cut from a sheet of substrate material, then bent outwards or “out of co-planarity,” such that the resulting microtips comprise projections from the substrate. In some embodiments, metal microtips are photochemically etched from a metal foil, and optionally electropolished while co-planar with the substrate or after bending out of co-planarity, for example if the bent microtip projections are still absent the drug or other substance. Photochemical etching is also referred to as photochemical machining (PCM) or simply “photoetching.” The process relies on UV-sensitive photoresist whereby unreacted photoresist is washed away to leave behind exposed areas of the substrate to be etched. Photochemical etching is often seen as an alternative to die- stamping, punching, laser cutting, waterjet cutting, and electrical discharge machining, although for purposes of the present disclosure, any of these precise milling processes are used singly or in any combination as appropriate. Half-etching refers to photochemical etching on only one side, rather than both sides, of a metal sheet substrate, such as to remove part of the thickness of the sheet substrate on one side only.

In some embodiments, plastic microtips are similarly die-cut or laser-cut into a plastic film, or alternatively, injection molded as a single molded part including both substrate and all the microtips.

Once bent nearly orthogonal, or at some other angle to the substrate sheet, microtips comprise projections from the substrate, remaining hingeably attached to the substrate at each of their proximal ends. In various embodiments, microtips are only partially cut into a precursor substrate sheet (e.g. incomplete cutting/material removal around the boundary of each microtip) such that each microtip remains attached to and coplanar with the substrate sheet. In this way, each microtip remains contiguous with and hingeably connected to the substrate material at an uncut portion of each microtip. This concept is more understandable by reference to the drawing figures, discussed below.

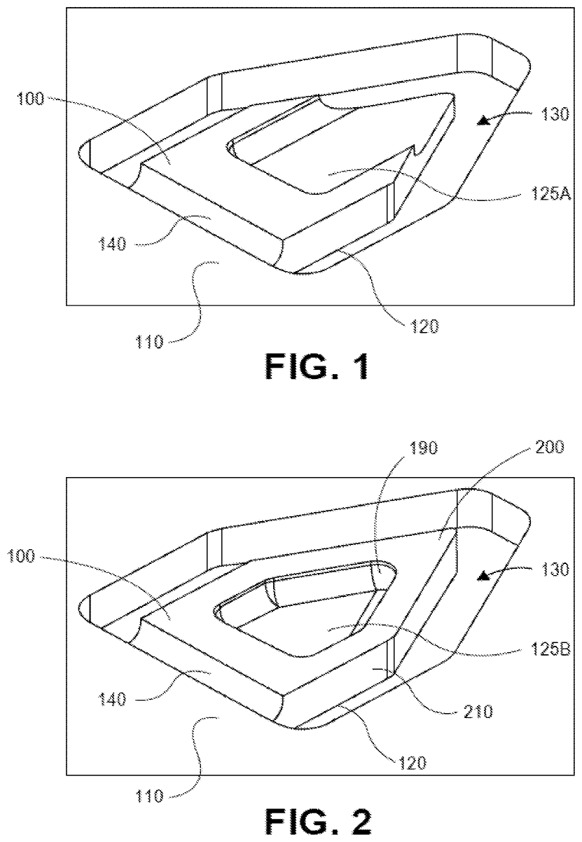

With reference now to FIGS. 1 and 2, two embodiments of an individual microtip in accordance to the present disclosure are illustrated in perspective view. In FIG. 1, substrate 110 is a sheet of material that is cut such that a discrete microtip outline 120 is formed. In some embodiments, the cutting process used to create a microtip 100 comprises simple die-cutting (e.g. stamping/punching) with the appropriately shaped punch, photochemical or electrochemical etching, or laser-cutting/ablation with a computer guided laser, or any combination of the above as mentioned, or any other method or combination of methods known to intricately cut metal or plastic sheeting. In some embodiments, the cutting process used to create a microtip is photochemical etching. As mentioned, microtips are optionally electropolished after any etching, cutting or ablation operation, before or after bending of the microtips out of co-planarity.

The shape of the microtip 100 is arrowhead-shaped as shown in FIGS. 1 and 2. In these examples, the thickness of the microtip 100 remains substantially the same as the thickness of the substrate 110, except where a depressed region (i.e. a reservoir) exists and at the hinged portion 140. In FIG. 2, microtip 100 comprises a straight edge 210 and a straight tip 200. In some embodiments, the reservoir is an open reservoir 125A, as shown in FIG. 1. In some embodiments, the reservoir is an enclosed reservoir 125B surrounded by a reservoir wall 190, as shown in FIG. 2. In some embodiments, the reservoir is a rectangular reservoir, a square reservoir, a triangular reservoir, a pentagonal reservoir, a hexagonal reservoir, an octagonal reservoir, a decagonal reservoir, an oval reservoir, or any other suitably shaped reservoir. In some embodiments, the reservoir is an open reservoir of any shape disclosed herein. In some embodiments, the reservoir is a closed reservoir of any shape disclosed herein. The reservoir 125A of FIG. 1, shown in microtip 100 as a depression on the microtip 100, is obtained by photochemical etching of a portion of the substrate 110 within the microtip outline 120, and as discussed, this open reservoir 125A is used as a reservoir for a substance, such as a drug, disposed on the microtip 100. In cases where enclosed reservoir 125B comprises a “well” within the boundaries of the microtip (e.g. as in FIG. 2), the enclosed reservoir 125B is produced by photochemical etching, laser ablation, or a stamping operation wherein a punch dents a portion of the substrate 110 to produce the depression. In some embodiments, such an operation is conducted simultaneously with a die-punch operation used to produce the microtip outlines. In various embodiments, photochemical etching is used to create both the outlines of the microtips in the substrate sheet and the reservoirs. For example, photochemical etching is used to remove portions of the substrate from both sides to create the microtip outlines, and from one side (half etching) to remove a portion of the thickness of the substrate at each microtip to fashion each reservoir.

In some embodiments, photochemical etching is used to create the outlines of the MicroArray in the substrate sheet. For example, photochemical etching is used to remove portions of the thickness of the substrate surrounding each MicroArray in a sheet of MicroArrays. In some embodiments, simple die-cutting (e.g. stamping/punching) with the appropriately shaped punch, electrochemical etching, or laser-cutting/ablation with a computer guided laser, or any combination of the above as mentioned, or any other method or combination of methods known to intricately cut metal sheeting is used to create the outlines of the MicroArray in the substrate sheet. In some embodiments, the outlines of the MicroArray in the substrate sheet are created before creating the outlines of the microtips, before creating the reservoirs, before depositing a substance in the reservoirs, or before bending the microtips out of planarity. In some embodiments, the outlines of the MicroArray in the substrate sheet facilitate die-cutting the MicroArrays with the appropriately shaped punch. In some embodiments, the MicroArrays in the substrate sheet are die-cut with the appropriately shaped punch, without the need to create a MicroArray outline. In some embodiments, the MicroArrays in the substrate sheet are photochemically etched and removed from the substrate sheet, without the need to create a MicroArray outline.

In other embodiments, a heated punch is used to soften/melt and finitely extrude a portion of the microtip shape in cases where the substrate 110 comprises plastic rather than metal. The machinery and conditions for the photochemical or electrochemical etching, the tooling for die-cutting, and/or the lasers for laser ablation, is chosen and configured accordingly depending on if the substrate 110 is metal or plastic, the precise type of metal or plastic, and the thickness of the material.

In some embodiments, the reservoir has a length of about 100 to 500 microns. In some embodiments, the reservoir has a length of about 100, 200, 300, 400, or 500 microns. In some embodiments, the reservoir has a length of about 400, 401, 402, 403, 404, 405, 406, 407, 408, 409, or 410 microns. In some embodiments, the reservoir has a width of about 50 to 300 microns. In some embodiments, the reservoir has a width of about 50, 100, 150, 200, 250, or 300 microns. In some embodiments, the reservoir has a width of about 200, 201, 202, 203, 204, 205, 206, 207, 208, 209, 210, 211, 212, 213, 214, 215, 216, 217, 218, 219, or 220 microns. In some embodiments, the reservoir has a depth of about 30 to 60 microns. In some embodiments, the reservoir has a depth of about 30, 35, 40, 45, 50, 55, or 60 microns. In some embodiments, the reservoir has a depth of about 35, 36, 37, 38, 39, 40, 41, 42, 43, 44, 45, 46, 47, 48, 49, 50, 51, 52, 53, 54, 55, 56, 57, 58, 59, or 60 microns.

With continued reference to FIG. 1, the outline of the microtip 100 further comprises a void 130 that is removed during the photochemical or electrochemical etching, die-punching or laser ablation processes used to create the microtip outline 120. In various embodiments, photochemical etching provides for rapid and high volume throughput, whereby microtip outlines and voids are obtained through the same process. A hinged portion 140 is left behind by avoiding complete material removal in this portion (the proximal end) of the microtip, which would detach the microtip 100 from substrate 110. The hinged portion 140 is the proximal end of microtip 100, the end where microtip 100 remains attached to the substrate 110. In this example, the hinged portion 140 is thinner than the thickness of the starting substrate 110. In some embodiments, the thickness of hinged portion 140 is purposely engineered to facilitate the eventual bending of the microtip 100 out of co-planarity. After the cutting process, and as shown in FIG. 1, a microtip 100 remains co-planar with the substrate 110. Thus, microtip 100 is in the x/y-plane of the substrate 110, and thus is not yet a projection emanating from substrate 110 until it is “stood up,” (i.e. bent across hinged portion 140 into the Z-plane to point approximately 90° or any other angle from the surface of the substrate 110). This bending aspect to produce microtip projections is discussed more thoroughly below.

Referring now to FIG. 2, a second embodiment of a microtip 100 according to the present disclosure is depicted. Each of the elements for microtip 100 corresponds to the elements discussed above in reference to microtip 100 in FIG. 1. As mentioned, the enclosed reservoir 125B in microtip 100 comprises a “well,” having discernable boundaries (walls) around all sides, and such a depression is etched or stamped into the metal substrate 110 or heat extruded with the appropriately shaped tool in the case of a plastic substrate sheet. As per the example in FIG. 2, a hinged portion 140 remains at the proximal end of the microtip 100 across which the microtip 100 is bent such that the microtip 100 then projects along the z-axis from the substrate 110. This bending aspect to produce microtip projections is discussed more thoroughly below.

In some embodiments, the microtip 100 has a length of about 400 to 800 microns. In some embodiments, the microtip 100 has a length of about 400, 450, 500, 550, 600, 650, 700, 750, or 800 microns. In some embodiments, the microtip 100 has a width of about 50 to 350 microns. In some embodiments, the microtip 100 has a width of about 50, 100, 150, 200, 250, 300, or 350 microns. In some embodiments, the microtip 100 has a width of about 300, 301, 302, 303, 304, 305, 306, 307, 308, 309, 310, 311, 312, 313, 314, 315, 316, 317, 318, 319, or 320 microns. In some embodiments, the hinged portion 140 of the microtip 100 has a depth of about 20 to 50 microns. In some embodiments, the hinged portion 140 of the microtip 100 has a depth of about 20, 25, 30, 35, 40, 45, or 50 microns. In some embodiments, the hinged portion 140 of the microtip 100 has a depth of about 20, 21, 22, 23, 24, 25, 26, 27, 28, 29, 30, 31, 32, 33, 34, 35, 36, 37, 38, 39, or 40 microns.

### Surface Roughness

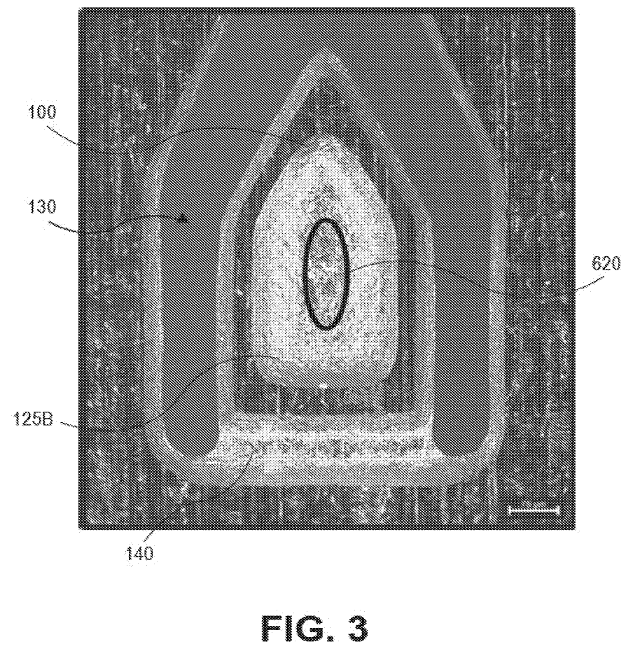

Referring now to FIG. 3, an image of a void 130 defining the outline of a microtip 100, wherein the microtip 100 comprises an enclosed reservoir 125B and a hinged portion 140, is shown. The image was captured using a Leica DVM6 microscope (Mid Magnification Objective: Leica PlanApo FOV 12.55). Microscope images of microtips are utilized to measure surface roughness. Surface roughness is a measure of the roughness of a given surface. Surface roughness of a microtip affects the frictional force between a microtip surface and a tissue. For example, a high frictional force causes tissue deflection, which hinders accurate microtip placement. In addition, increased surface roughness increases insertion and retraction forces, which influence patient discomfort and pain. A rougher microtip surface leads to a greater insertion force, a greater retraction force, and consequently greater patient pain and discomfort.

In some embodiments, surface roughness is determined using the Surface Imaging and Metrology Software Leica Map. A surface roughness parameter (Sa), which is the arithmetic mean of the surface height of all points of a certain region, is determined based on images of the surface of a microtip. Accurate measurement of surface roughness of curved surfaces is difficult to achieve since it requires complicated post-processing to remove the effect of the curvature of the object. Not doing so leads to large variation of surface roughness values of the same or similar areas. In some embodiments, small regions of interests (ROIs) (with dimensions of 10 μm×10 μm, for example) in images with a magnification of approximately 380× are drawn in the center of needle reservoirs where the reservoir surface has the least curvature. In some embodiments, multiple ROIs, for example, four ROIs, are drawn per reservoir. FIG. 3B shows an exemplary ROI 620.

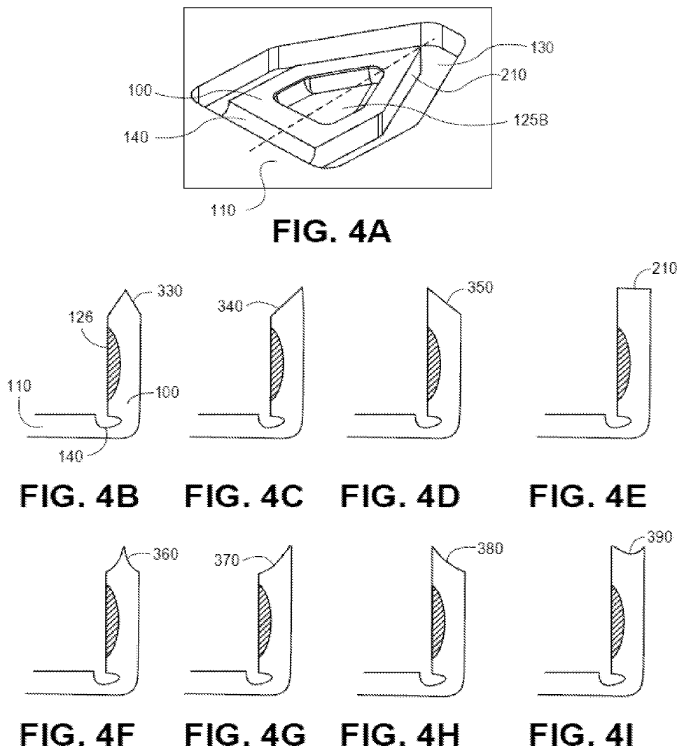

Referring now to FIGS. 4A-I, various embodiments of a microtip 100 comprising different types of finished edges are illustrated. The finished edges of the microtip define its tip geometry. Needle tip geometry affects skin penetration force and therefore, also affects the pain experienced by patients during needle insertion. Microtips with beveled edges (also referred to as a feathered edge) typically have sharper tips compared to microtips with straight edges. Feathered or beveled-edged microtips require less penetration force than microtips lacking feathered or beveled edges and consequently, patients experience less pain during insertion of feathered or beveled-edged microtips.

FIG. 4A illustrates a microtip 100 attached to a substrate 110 and comprising a hinged portion 140, an enclosed reservoir 125B, and a straight edge 210. In some embodiments, the microtip 100 comprises a non-beveled edge. In some embodiments, the straight edge 210 is a non-beveled edge. The straight edge 210 is perpendicular to the top surface of the microtip comprising the reservoir 125B and is perpendicular to the bottom surface of the microtip facing the void 130. In some embodiments, the angles between the top surface of the microtip and the straight edge 210 and between the bottom surface of the microtip and the straight edge 210 are 90 degrees. Furthermore, FIG. 4A depicts a dotted line intersecting the microtip longitudinally at its center to serve as a reference for the cross-sectional views presented in FIGS. 4B-I. In other words, FIGS. 4B-I are representative illustrations of cross-sectional views of a microtip 100 that has been dissected longitudinally at its center, as shown by the dotted line in FIG. 4A. FIGS. 4B-I are viewed from the position of the plane dissecting the microtip 100, as defined by the dotted line in FIG. 4A.

In some embodiments, the microtip 100 comprises a beveled edge. Any suitable type of beveled edge is contemplated as an element of the microtips disclosed herein. As shown in FIGS. 4B-D and 4F-I, in some embodiments, the beveled edge is a double beveled edge 330, a top beveled edge 340, a bottom beveled edge 350, a double concave beveled edge 360, a top concave beveled edge 370, a bottom concave beveled edge 380, or a concave beveled edge 390. In some embodiments, the beveled edge is a double beveled edge 330. In some embodiments, the beveled edge is a top beveled edge 340. In some embodiments, the beveled edge is a bottom beveled edge 350. In some embodiments, the beveled edge is a double concave beveled edge 360. In some embodiments, the beveled edge is a top concave beveled edge 370. In some embodiments, the beveled edge i a bottom concave beveled edge 380, or a concave beveled edge 390.

In some embodiments, the microtip 100 comprises a double beveled edge 330. The double beveled edge 330 comprises a first bevel and a second bevel that intersect, as shown by the two sloped lines intersecting and forming a vertex in FIG. 4B. This type of bevel is known as a “X” bevel because when two materials with this type of bevel are put side by side, their profile looks like the letter “X.” The first bevel of the double beveled edge 330 originates from the bottom surface of the microtip 100 (i.e. the side of the microtip that faces the void 130) and the second bevel of the double beveled edge 330 originates from the top surface of microtip 100 (i.e. the side of the microtip that contains the reservoir), as shown in FIG. 4B. Both the first bevel and the second bevel end intersect.

In some embodiments the angle between the bottom surface of the microtip and the first bevel in a double beveled edge 330 is less than 90 degrees. In some embodiments the angle between the bottom surface of the microtip and the first bevel in a double beveled edge 330 ranges between from about 0 to about 89 degrees. In some embodiments the angle between the bottom surface of the microtip and the first bevel in a double beveled edge 330 is about 45 degrees. In some embodiments, the angle between the bottom surface of the microtip and the first bevel in a double beveled edge 330 is about 45, 50, 55, 60, 65, 70, 75, 80, or 85 degrees. In some embodiments the angle between the top surface of the microtip and the second bevel in a double beveled edge 330 is less than 90 degrees. In some embodiments the angle between the top surface of the microtip and the second bevel in a double beveled edge 330 ranges between from about 0 to about 89 degrees. In some embodiments the angle between the top surface of the microtip and the second bevel in a double beveled edge 330 is about 45 degrees. In some embodiments, the angle between the top surface of the microtip and the second bevel in a double beveled edge 330 is about 45, 50, 55, 60, 65, 70, 75, 80, or 85 degrees.

In some embodiments, the angle between the bottom surface of the microtip and the first bevel in a double beveled edge 330 is equal to the angle between the top surface of the microtip and the second bevel. In some embodiments, the angle between the bottom surface of the microtip and the first bevel is not equal to the angle between the top surface of the microtip and the second bevel in a double beveled edge 330. In some embodiments, the angle between the bottom surface of the microtip and the first bevel is greater than the angle between the top surface of the microtip and the second bevel in a double beveled edge 330. In some embodiments, the angle between the bottom surface of the microtip and the first bevel is less than the angle between the top surface of the microtip and the second bevel in a double beveled edge 330.

In some embodiments, the microtip 100 comprises a top beveled edge 340. The top beveled edge 340 runs from the top surface of the microtip (i.e. the surface of the microtip comprising the reservoir) to the bottom edge of the microtip. The top beveled edge 340 is commonly known as the a “V” bevel because when two materials with this type of bevel are put side by side, their profile looks like the letter “V.” In some embodiments, the microtip 100 comprises a bottom beveled edge 350. The bottom beveled edge 350 is basically the inverse of the top beveled edge 340. In other words, the bottom beveled edge 350 runs from the bottom surface of the microtip (i.e. the surface of the microtip facing the void) to the top edge of the microtip.

In some embodiments, the microtip 100 comprises a double concave beveled edge 360. The double concave beveled edge 360 is similar in structure to the double beveled edge 330 in that the double concave beveled edge 360 comprises a first bevel and a second bevel that intersects and forms a vertex. However, the first bevel and the second bevel are concave. In some embodiments, the microtip 100 comprises a top concave beveled edge 370. The top concave beveled edge 370 is similar in structure to the top beveled edge 340 in that the top concave beveled edge 370 comprises a bevel that runs from the top surface of the microtip (i.e. the surface of the microtip comprising the reservoir) to the bottom edge of the microtip. However, unlike the top beveled edge 340, the top concave beveled edge 370 is a concave bevel. In some embodiments, the microtip 100 comprises a bottom concave beveled edge 380. The top concave beveled edge 380 is similar in structure to the bottom beveled edge 350 in that the bottom concave beveled edge 380 comprises a bevel that runs from the bottom surface of the microtip (i.e. the surface of the microtip facing the void) to the top edge of the microtip. However, unlike the bottom beveled edge 350, the top concave beveled edge 380 is a concave bevel. In some embodiments, the microtip 100 is a concave beveled edge 390. The concave beveled edge 390 results in a microtip comprising two distinct tips.

In some embodiments, the microtip 100 comprising a straight edge 210 has a length of about 500 to 700 microns. In some embodiments, the microtip 100 has a length of about 500, 550, 600, 650, or 700 microns. In some embodiments, the microtip 100 has a length of about 580, 581, 582, 583, 584, 585, 586, 587, 588, 589, 590, 591, 592, 593, 594, 595, 596, 597, 598, 599, or 600 microns.

In some embodiments, the microtip 100 comprising a beveled edge (i.e. an edge such as the edges depicted in FIGS. 4B-D and 4-F-I) has a length of about 600 to 800 microns. In some embodiments, the microtip 100 comprising an altered edge has a length of about 600, 650, 700, 750, or 800 microns. In some embodiments, the microtip 100 comprising an altered edge has a length of about 690, 691, 692, 693, 694, 695, 696, 697, 698, 699, 700, 701, 702, 703, 704, 705, 706, 707, 708, 709, 710, 711, 712, 713, 714, or 715 microns.

### MicroArrays

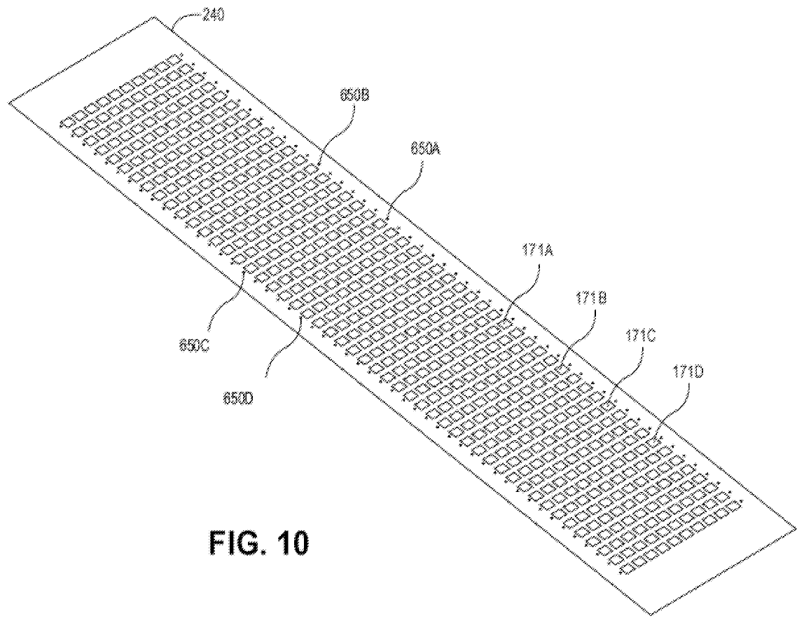

As discussed, MicroArrays comprise more than one microtip, such as comprising a plurality of microtips. To produce an array, a plurality of outlines of microtips, such as multiples of those shown individually in FIG. 1 and FIG. 2, are created on a single substrate sheet (e.g., FIG. 10), by photochemically or electrochemically etching, die-cutting, or laser-cutting the plurality of microtips all at once with the appropriate systems, tooling, or computer-guided laser, to cut the many outlines, or by combining methods as appropriate. In some embodiments, a planar metal substrate is photochemically etched to produce rows and columns of individual MicroArrays, each MicroArray comprising a plurality of microtips, and each microtip comprising a half-etched reservoir and a hinge portion 140 suitable for bending the microtips from the X, Y-plane into Z-plane (as shown in FIGS. 1 and 2). In some embodiments, any suitable number of MicroArrays is present in each row (x-axis) or each column (y-axis) of a substrate sheet. For example, in some embodiments, a MicroArray sheet comprises 10 MicroArrays per row and 50 MicroArrays per column, to produce a MicroArray Sheet comprising 500 individual MicroArrays (e.g., FIG. 10).

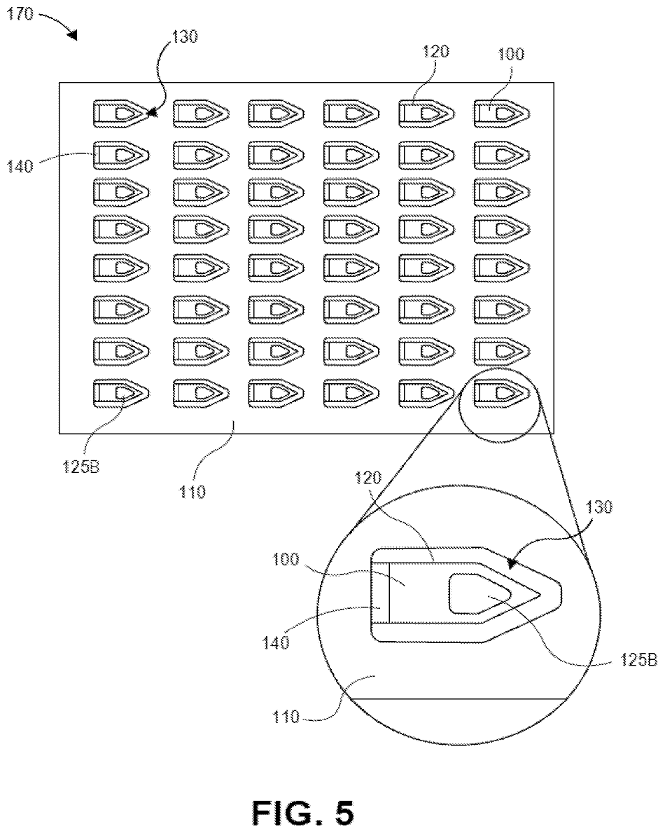

With reference now to FIG. 5, an excised, empty, flat MicroArray 170 is depicted, along with a detailed portion of one of the microtips. This exemplary array happens to have a 6×8 grid of microtips cut from substrate 110 (48 total microtips), although any number, and any arrangement of microtips are provided on a substrate to produce any configuration for a MicroArray, such as depending on the size of the substrate sheet, the shape of the microtips, end use considerations, (e.g. the nature of the substance to be delivered), and the desired density of microtips per square surface area, amongst other considerations. In one non-limiting example, a MicroArray comprises a microtip density of about 650 microtip projections per 1 cm² of substrate surface area.

With continued reference to FIG. 5 and the magnified detail therein, each microtip 100 includes a hinged portion at the proximal end, as per the examples in FIGS. 1 and 2. Also, the substrate 110 is seen to have a void 130 that defines the microtip outline 120, also similar to the examples in FIGS. 1 and 2. Each microtip 100 in the excised, empty, flat Micro Array 170 is arrow-shaped, and each range in length from about 10-1000 microns in length (e.g., 400 to 500 microns, from proximal hinged portion to pointed distal tip), about 10-1000 microns in width (e.g. about 175 microns to about 250 microns), and about 0.5 micron to about 200 microns in thickness (e.g. 25 to 75 microns), and similar or identical in thickness to the substrate 110. As mentioned, and discounting the three-dimensionality afforded by the optional reservoirs, the MicroArray 170 remains generally planar, as each of the microtips remain co-planar with the substrate 110 from which they were cut.

Still with reference to FIG. 5 and specifically with reference to the magnified portion, each microtip 100 further comprises an enclosed reservoir 125B that holds a substance to be delivered by the MicroArray. Reservoirs comprise any shape or size, (e.g. conical, pyramidal, cubical, etc.). In some embodiments, such reservoirs have a depth of about 0.5 to 100 microns, depending on the desired amount of substance loading anticipated for each microtip 100 and factoring in constraints of thickness of the substrate 110. As discussed, each reservoir on each of the microtips in the MicroArray is created simultaneously, such as by photochemical half etching of some of the thickness of the substrate 110, by denting the substrate 110 in each of these regions with the appropriately shaped and dimensioned tooling, or by extruding a plastic substrate in these regions using heated tools that soften/melt while forming each reservoir. In some embodiments, the volume of each reservoir ranges from about 0.1 nL to about 1 μL, or about 0.1 nL to about 5 nL, or from about 1 nL to about 2 nL, or from about 1 nL to about 10 nL, from about 2 nL to about 3 nL, from about 3 nL to about 4 nL, from about 4 nL to about 5 nL, from about 5 nL to about 6 nL, from about 6 nL to about 7 nL, from about 7 nL to about 8 nL, from about 8 nL to about 9 nL, from about 9 nL to about 10 nL, from about 10 nL to about 15 nL, from about 15 nL to about 20 nL, from about 20 nL to about 25 nL, from about 25 nL to about 30 nL, from about 30 nL to about 35 nL, or from about 35 nL to about 40 nL. Each of the reservoirs on each of the microtips are then filled/loaded with a substance to be delivered to a patient in need thereof.

In various embodiments, a step of electropolishing (also known as electrochemical polishing or electrolytic polishing) follows the step of photochemical etching of the microtips and reservoirs into the substrate. In other aspects, electropolishing follows any other type of etching, cutting, or ablation process as needed to precisely finish the microtip and reservoir shapes and dimensions. In some embodiments, electropolishing is used to remove unwanted material from the substrate after the etching or cutting process. In some embodiments, electropolishing precedes or subsequently follows the bending of the cut microtips out of co-planarity, provided that for the latter, the drug or other substance is not yet present on the microtips. In certain aspects, wherein a substance, e.g. a drug, is already loaded onto microtips (e.g. into their respective reservoirs) while the microtips remain coplanar with the substrate, it is desirable to electropolish the microtips after etching the microtip outlines and reservoirs but prior to loading of the desired substance into the reservoirs. In some embodiments, the procedure is to etch the microtip outlines and reservoirs into the substrate, electropolish, rinse and clean up the substrate, load the desired substance into the reservoirs, and then bend the microtips out of co-planarity. In other embodiments, microtips and optionally reservoirs are etched into the substrate, the microtips are bent out of co-planarity into projections, the microtips are electropolished, cleaned and rinsed, and then the projections are roll- or dip-coated in a drug or other substance.

In various embodiments, the 2D array of co-planar microtips (i.e. just subsequent to photochemical etching of the microtip outlines and the associated reservoirs) is electropolished, rinsed with deionized (DI) water, cleaned in a sonic cleaner (e.g. from Steris Corp.), drained, dried, heat sterilized, and then cooled to room temperature prior to dispensing of the substance into the reservoirs. In some embodiments, after drying, a lubricant is added by dipping the 2D array into a 2-5% solution of MDX4-4159 (a medical grade dispersion available from Dow Corning) for 5-15 minutes, then draining, drying and heat sterilizing as mentioned. Addition of a lubricant to microtips is believed to be heretofore unknown and is likely to aid penetration of the microtips into the skin of the subject.

### Loading the Microtips with a Substance to be Delivered

As discussed, in some embodiments, each of the microtips in a MicroArray is coated or otherwise “loaded” with a substance to be delivered to a patient. Microtips having substantially a two-dimensional structure (e.g. flat, arrow-shaped pieces of metal) are simply dipped into the substance, removed, and, if necessary, dried under ambient or the appropriate heat/vacuum conditions. To coat microtips with a flowable substance, any coating method is used, e.g. for example gravure coating, roll-coating, dip, spray, and combinations thereof. In some embodiments, substances of other physical form (e.g. solid) are sublimed and deposited on microtips, suspended in a liquid and coated as per above, or powder coated through the appropriate nebulizer. In some embodiments, a coating of a substance on a microtip comprises any thickness, for example, from about 1 micron to about 100 microns thick, and is located on only a select portion of the microtips (the distal end portion, for example), or on most or all of each needle. In some embodiments, a coating of a substance is allowed to dry, whereby various volatile ingredients in the composition vaporize. Alternatively, in other embodiments, coated microtips arrays are subsequently subjected to freeze-drying, heating, vacuum, or an autoclave to dry, polymerize or even chemically change the coating of substance on each microtip.

The weight of substance coated on each microtip depends on a number of factors, such as, for example, the nature of the substance, (e.g. neat API versus a chemical composition comprising an API), the volatility, stability, etc. of the substance, nature of the API in a composition, viscosity of the substance, the available surface area of the microtips for coating, the temperature of the microtips during coating, amongst other considerations. In some embodiments, as little as 1 μg of substance is loaded onto all of the microtips in an array, combined. In other variations, from about 1 μg to about 1 mg of substance is loaded onto each microtip in a MicroArray. Substances are layered on as desired. For example, a first drug, then a stabilizer, and finally a second drug, is added in sequence to each microtip.

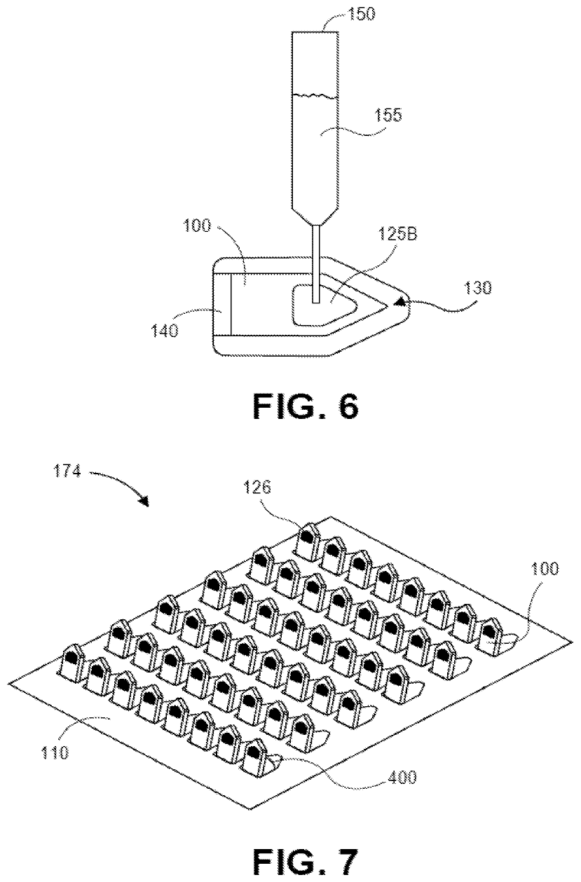

In some embodiments, each microtip of a MicroArray further comprises a depressed region or a reservoir that is filled with a substance, (e.g., FIGS. 7-8 and 14). FIG. 6 illustrates the filling of an empty reservoir in a microtip 100 with a substance 155 and using a nozzle 150. With reference to FIG. 6, a nozzle 150 is shown charged with substance 155, and such a device is used to pipette, deposit, inject, introduce, print, or eject a small amount of substance 155 into the each empty reservoir of each microtip 100 within an array. In some embodiments, nozzle 150 is a pipette, a print nozzle, a microfluidic dispensing device nozzle (i.e. a microfluidic dispense nozzle), an automatic liquid dispenser nozzle, an automatic liquid handler nozzle, or a syringe.

In some embodiments, quantitative picoliter and nanoliter automated microfluidic dispensing systems are utilized to dispense a substance into each microtip reservoir present in a MicroArray. The BioDot AD 1520 system, available from BioDot Inc., Irving, California, is an example of an automated microfluidic dispensing system. Such microfluidic dispensing instruments are appropriate for large scale manufacturing, and are able to dispense as little as 1 nL with an x-, y-, z-axis positional accuracy of about 10 microns, such that each microtip reservoir is accurately located and the appropriate amount of sample dispensed with the speed required for the volumes disclosed herein. Other microfluidic instruments are capable dispense down to 200 picoliters. In some embodiments, each microtip reservoir in a MicroArray is filled with about 0.1 nL to about 1 μL, about 0.1 nL to about 5 nL, about 1 nL to about 2 nL, about 3 nL to about 4 nL, about 5nL to about 10 nL, about 10 nL to about 20 nL, from about 0.1 nL to about 5 nL, from about 2 nL to about 3 nL, from about 4 nL to about 5 nL, from about 5 nL to about 6 nL, from about 6 nL to about 7 nL, from about 7 nL to about 8 nL, from about 8 nL to about 9 nL, from about 9 nL to about 10 nL, from about 10 nL to about 15 nL, from about 15 nL to about 20 nL, from about 20 nL to about 25 nL, from about 25 nL to about 30 nL, from about 30 nL to about 35 nL, or from about 35 nL to about 40 nL, about 20 nL to about 30 nL, or about 30 nL to about 40 nL of a substance.

The differences between precise loading of a substance into a reservoir of a microtip and the dipping/roll-coating of a generally two-dimensional microtip in a substance should not be underestimated. As mentioned, dipping and roll-coating as well as other methods such as gas-jet drying, spray drying, and electrohydrodynamic atomization (EHDA) processes are inefficient, wasteful processes, but are, for the most part, the only practical methods for loading drugs or other substances onto microtips that are already bent into orthogonal projections or onto microtips that are already created in vertical orientations. In contrast with the precise microtip loading methods described herein, the dipping, roll-coating, gas-jet drying, spray drying, and EHDA methods are not amenable to be scaled up into a consistently repeatable and yield-efficient manufacturing process. For example, some disadvantages of these methods include: lengthy process steps that require high temperatures (i.e. preparation of the formulation in a hot stage), addition of extra excipients to overcome surface tension issues, overspreading of substance onto microtip and microtip base due to surface tension, addition of surfactants, lengthy substance drying times, non-uniform coating, gravitational and low surface tension spreading of substance on microtips, and inability to coat microtips with substances that have a low electric conductivity (e.g. with the EHDA method). Vertically oriented projections cannot be drug loaded by precision dispensing. In some embodiments, the processes in accordance to the present disclosure comprise the precision dispensing of a substance (e.g. a vaccine) into the reservoirs of the microtips while the microtips remain co-planar with the substrate (i.e., prior to bending upright). In some embodiments, the processes described herein are scalable manufacturing processes. In some embodiments, the processes described herein comprise steps that are not executed at temperatures above ambient temperature. In some embodiments, the substances loaded onto the microtips described herein do not comprise a surfactant. In some embodiments, the substances loaded onto the microtips described herein do not comprise an excipient to counteract issues arising from gravity and/or surface tension, such as unwanted spreading of substance, unwanted overspreading of substance, and/or non-uniformly coated microtips. In some embodiments, the substances loaded onto the microtips described herein have low electric conductivity.

In some embodiments, the microfluidic dispensing device comprises a plurality of microfluidic dispense nozzles (i.e., multi-channel microfluidic dispensing device), with each microfluidic dispense nozzle configured to dispense a sample fluid into a microtip reservoir of a MicroArray. In some embodiments, a substrate comprising rows and columns of photoetched MicroArrays (e.g. the MicroArray shect 240 shown in FIG. 10), further comprises a plurality of fiducial markers including a first fiducial marker 650A, a second fiducial marker 650B, a third fiducial marker 650C, a fourth fiducial marker 650D, etc., which facilitates the accurate location of individual MicroArrays within the rows (i.e., x-axis) and columns (i.e., y-axis) of the substrate by a surface mount technology (SMT) component placement system, also known as a “pick-and-place” (P&P) system, for example.

SMT systems are robotic machines comprising robotic arms that are used to pick-up, handle, and place specific device components with a high precision and at a high speed. SMT systems typically use pneumatic suction cups to pick-up, handle and place specific device components. The pneumatic suction cups are attached to a device that enables the suction cup to be rotated in three dimensions. Furthermore, SMT systems are operably connected to an optical system comprising a plurality of cameras and a computing device. A first camera photographs fiducial markers to accurately measure the position of the device receiving a component on a conveyor belt. A second camera attached to the robotic arm photographs the fiducial markers to accurately measure the position of the component being delivered to the device on the conveyor belt.

In some embodiments, the multi-channel microfluidic dispensing device is operably linked to an SMT system comprising a robotic arm, pneumatic suction cups, and an optical system that utilize the spatial organization of the fiducial markers to accurately align the dispensing nozzles over a row of MicroArrays, enabling the dispensing of a substance into each microtip reservoir at high speeds.

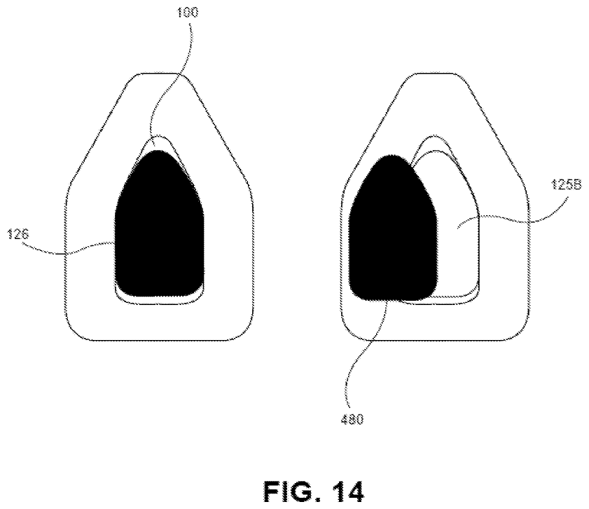

In some embodiments, the substance loaded into the microtip reservoirs of a MicroArray are formulated as a sugar glass or sugar crystal. In some embodiments, the sugar glass comprises sucralose, glucose, galactose, fructose, trehalose, maltose, or a combination thereof. In some embodiments, the sugar glass is a trehalose sugar glass. In some embodiments, the sugar glass is a sucrose and trehalose sugar glass. The formation and properties of sugar glasses are well known in the art and any suitable sugar glass is contemplated for use with the microtips and MicroArrays disclosed herein. In some embodiments, a vaccine is trapped in a sugar glass. In contrast to conventional vaccines in suspension, vaccines immobilized within a sugar glass are able to withstand degradation at relatively high temperatures for extended periods of time (e.g. months). The ability to preserve vaccines without the need for refrigeration (i.e. cold chain for storage and transport) is of great importance in third world countries and/or areas in which electricity and/or refrigeration vehicles are not readily available. FIG. 14 shows a microtip 100 comprising a filled reservoir 126 loaded with a sugar glass vaccine 480. As shown in FIG. 14, the sugar glass vaccine 480 dries and becomes a solid that is easily removed from the enclosed reservoir 125B.

### Bending Microtips to Form Projections

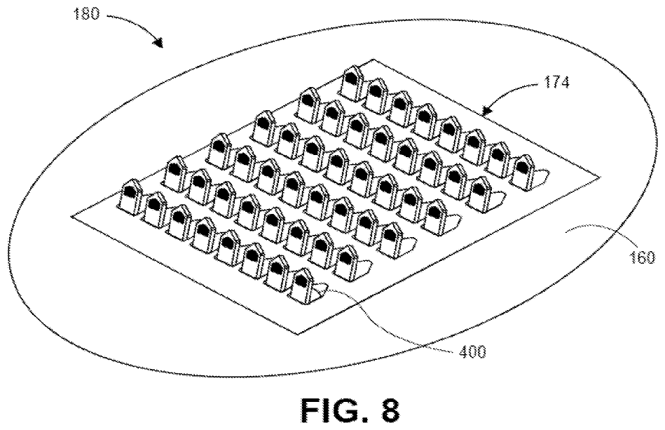

As discussed above, microtips etched/cut into a substrate sheet are subsequently bent out of plane such that each microtip becomes a projection emanating from the substrate sheet. Referring now to FIG. 7, a cut microtip 100 is bent into a projection pointing at an angle 400 from the surface of substrate 110. The angle 400 between the bent microtip and the surface of the substrate 110 is depicted by an arc, as shown in FIGS. 7 and 8. In some embodiments, the angle 400 between the bent microtip and the surface of the substrate 110 ranges from about 45 to about 135 degrees.

In some embodiments, the angle 400 between the bent microtip and the surface of the substrate 110 is about 45 degrees. In some embodiments, the angle 400 between the bent microtip and the surface of the substrate 110 is about 46 degrees. In some embodiments, the angle 400 between the bent microtip and the surface of the substrate 110 is about 47 degrees. In some embodiments, the angle 400 between the bent microtip and the surface of the substrate 110 is about 48 degrees. In some embodiments, the angle 400 between the bent microtip and the surface of the substrate 110 is about 49 degrees. In some embodiments, the angle 400 between the bent microtip and the surface of the substrate 110 is about 50 degrees. In some embodiments, the angle 400 between the bent microtip and the surface of the substrate 110 is about 51 degrees. In some embodiments, the angle 400 between the bent microtip and the surface of the substrate 110 is about 52 degrees. In some embodiments, the angle 400 between the bent microtip and the surface of the substrate 110 is about 53 degrees. In some embodiments, the angle 400 between the bent microtip and the surface of the substrate 110 is about 54 degrees. In some embodiments, the angle 400 between the bent microtip and the surface of the substrate 110 is about 55 degrees. In some embodiments, the angle 400 between the bent microtip and the surface of the substrate 110 is about 56 degrees. In some embodiments, the angle 400 between the bent microtip and the surface of the substrate 110 is about 57 degrees. In some embodiments, the angle 400 between the bent microtip and the surface of the substrate 110 is about 58 degrees. In some embodiments, the angle 400 between the bent microtip and the surface of the substrate 110 is about 59 degrees. In some embodiments, the angle 400 between the bent microtip and the surface of the substrate 110 is about 60 degrees. In some embodiments, the angle 400 between the bent microtip and the surface of the substrate 110 is about 61 degrees. In some embodiments, the angle 400 between the bent microtip and the surface of the substrate 110 is about 62 degrees. In some embodiments, the angle 400 between the bent microtip and the surface of the substrate 110 is about 63 degrees. In some embodiments, the angle 400 between the bent microtip and the surface of the substrate 110 is about 64 degrees. In some embodiments, the angle 400 between the bent microtip and the surface of the substrate 110 is about 65 degrees. In some embodiments, the angle 400 between the bent microtip and the surface of the substrate 110 is about 66 degrees. In some embodiments, the angle 400 between the bent microtip and the surface of the substrate 110 is about 67 degrees. In some embodiments, the angle 400 between the bent microtip and the surface of the substrate 110 is about 68 degrees. In some embodiments, the angle 400 between the bent microtip and the surface of the substrate 110 is about 69 degrees. In some embodiments, the angle 400 between the bent microtip and the surface of the substrate 110 is about 70 degrees. In some embodiments, the angle 400 between the bent microtip and the surface of the substrate 110 is about 71 degrees. In some embodiments, the angle 400 between the bent microtip and the surface of the substrate 110 is about 72 degrees. In some embodiments, the angle 400 between the bent microtip and the surface of the substrate 110 is about 73 degrees. In some embodiments, the angle 400 between the bent microtip and the surface of the substrate 110 is about 74 degrees. In some embodiments, the angle 400 between the bent microtip and the surface of the substrate 110 is about 75 degrees. In some embodiments, the angle 400 between the bent microtip and the surface of the substrate 110 is about 76 degrees. In some embodiments, the angle 400 between the bent microtip and the surface of the substrate 110 is about 77 degrees. In some embodiments, the angle 400 between the bent microtip and the surface of the substrate 110 is about 78 degrees. In some embodiments, the angle 400 between the bent microtip and the surface of the substrate 110 is about 79 degrees. In some embodiments, the angle 400 between the bent microtip and the surface of the substrate 110 is about 80 degrees. In some embodiments, the angle 400 between the bent microtip and the surface of the substrate 110 is about 81 degrees. In some embodiments, the angle 400 between the bent microtip and the surface of the substrate 110 is about 82 degrees. In some embodiments, the angle 400 between the bent microtip and the surface of the substrate 110 is about 83 degrees. In some embodiments, the angle 400 between the bent microtip and the surface of the substrate 110 is about 84 degrees. In some embodiments, the angle 400 between the bent microtip and the surface of the substrate 110 is about 85 degrees. In some embodiments, the angle 400 between the bent microtip and the surface of the substrate 110 is about 86 degrees. In some embodiments, the angle 400 between the bent microtip and the surface of the substrate 110 is about 87 degrees. In some embodiments, the angle 400 between the bent microtip and the surface of the substrate 110 is about 88 degrees. In some embodiments, the angle 400 between the bent microtip and the surface of the substrate 110 is about 89 degrees. In some embodiments, the angle 400 between the bent microtip and the surface of the substrate 110 is about 90 degrees. In some embodiments, the angle 400 between the bent microtip and the surface of the substrate 110 is about 91 degrees. In some embodiments, the angle 400 between the bent microtip and the surface of the substrate 110 is about 92 degrees. In some embodiments, the angle 400 between the bent microtip and the surface of the substrate 110 is about 93 degrees. In some embodiments, the angle 400 between the bent microtip and the surface of the substrate 110 is about 94 degrees. In some embodiments, the angle 400 between the bent microtip and the surface of the substrate 110 is about 95 degrees. In some embodiments, the angle 400 between the bent microtip and the surface of the substrate 110 is about 96 degrees. In some embodiments, the angle 400 between the bent microtip and the surface of the substrate 110 is about 97 degrees. In some embodiments, the angle 400 between the bent microtip and the surface of the substrate 110 is about 98 degrees. In some embodiments, the angle 400 between the bent microtip and the surface of the substrate 110 is about 99 degrees. In some embodiments, the angle 400 between the bent microtip and the surface of the substrate 110 is about 100 degrees. In some embodiments, the angle 400 between the bent microtip and the surface of the substrate 110 is about 101 degrees. In some embodiments, the angle 400 between the bent microtip and the surface of the substrate 110 is about 102 degrees. In some embodiments, the angle 400 between the bent microtip and the surface of the substrate 110 is about 103 degrees. In some embodiments, the angle 400 between the bent microtip and the surface of the substrate 110 is about 104 degrees. In some embodiments, the angle 400 between the bent microtip and the surface of the substrate 110 is about 105 degrees. In some embodiments, the angle 400 between the bent microtip and the surface of the substrate 110 is about 106 degrees. In some embodiments, the angle 400 between the bent microtip and the surface of the substrate 110 is about 107 degrees. In some embodiments, the angle 400 between the bent microtip and the surface of the substrate 110 is about 108 degrees. In some embodiments, the angle 400 between the bent microtip and the surface of the substrate 110 is about 109 degrees. In some embodiments, the angle 400 between the bent microtip and the surface of the substrate 110 is about 110 degrees. In some embodiments, the angle 400 between the bent microtip and the surface of the substrate 110 is about 111 degrees. In some embodiments, the angle 400 between the bent microtip and the surface of the substrate 110 is about 112 degrees. In some embodiments, the angle 400 between the bent microtip and the surface of the substrate 110 is about 113 degrees. In some embodiments, the angle 400 between the bent microtip and the surface of the substrate 110 is about 114 degrees. In some embodiments, the angle 400 between the bent microtip and the surface of the substrate 110 is about 115 degrees. In some embodiments, the angle 400 between the bent microtip and the surface of the substrate 110 is about 116 degrees. In some embodiments, the angle 400 between the bent microtip and the surface of the substrate 110 is about 117 degrees. In some embodiments, the angle 400 between the bent microtip and the surface of the substrate 110 is about 118 degrees. In some embodiments, the angle 400 between the bent microtip and the surface of the substrate 110 is about 119 degrees. In some embodiments, the angle 400 between the bent microtip and the surface of the substrate 110 is about 120 degrees. In some embodiments, the angle 400 between the bent microtip and the surface of the substrate 110 is about 121 degrees. In some embodiments, the angle 400 between the bent microtip and the surface of the substrate 110 is about 122 degrees. In some embodiments, the angle 400 between the bent microtip and the surface of the substrate 110 is about 123 degrees. In some embodiments, the angle 400 between the bent microtip and the surface of the substrate 110 is about 124 degrees. In some embodiments, the angle 400 between the bent microtip and the surface of the substrate 110 is about 125 degrees. In some embodiments, the angle 400 between the bent microtip and the surface of the substrate 110 is about 126 degrees. In some embodiments, the angle 400 between the bent microtip and the surface of the substrate 110 is about 127 degrees. In some embodiments, the angle 400 between the bent microtip and the surface of the substrate 110 is about 128 degrees. In some embodiments, the angle 400 between the bent microtip and the surface of the substrate 110 is about 129 degrees. In some embodiments, the angle 400 between the bent microtip and the surface of the substrate 110 is about 130 degrees. In some embodiments, the angle 400 between the bent microtip and the surface of the substrate 110 is about 131 degrees. In some embodiments, the angle 400 between the bent microtip and the surface of the substrate 110 is about 132 degrees. In some embodiments, the angle 400 between the bent microtip and the surface of the substrate 110 is about 133 degrees. In some embodiments, the angle 400 between the bent microtip and the surface of the substrate 110 is about 134 degrees. In some embodiments, the angle 400 between the bent microtip and the surface of the substrate 110 is about 135 degrees.

In some embodiments, the angle 400 between the bent microtip and the surface of the substrate 110 is about 90 degrees. Other angles of projection are within the scope of the invention, such as, for example, any angle from about 50° to about 90°. To this end, microtips are bent through an arc from between 0° to 90°, hinging on the proximal hingeable portion on each microtip as discussed. It should be recognized that in the bending process, in some embodiments, microtips are “overextended,” meaning any microtip is bent through an arc measuring greater than 90°, such that the resulting angle between each microtip projection and the plane of the substrate ends up less than 90°. For example, each microtip is bent across the hingeable portion of the microtip in an arc of about 100°, leaving the microtips to project at an angle of 80° relative to the planar substrate surface.

Temporarily referring back to the microtip example in FIG. 1, and the corresponding discussion above, it was mentioned that a hinged portion 140 is engineered on each microtip, for example by photochemical etching or laser ablation of some of the thickness of the substrate. In particular the thickness of hinged portion 140 is purposely engineered to facilitate the bending of the microtip 100 out of co-planarity. In some embodiments, creating a thinner hinged portion 140 mitigates distortions to the shape of the microtip 100 when microtip 100 is bent across this hinged portion 140. In various embodiments, heating the cut substrate promotes a softening of the substrate material at only these thinner hingeable portions, thus facilitating bending of microtip 100 out of co-planarity, without damaging the overall MicroArray or the shapes of the microtips. In cases of a plastic substrate, the thinner, hinged portion 140 is pre-softened to facilitate bending of the microtip. In various embodiments, the substrate 110 comprises a thermoplastic material, which, when heated near its transition temperature, softens selectively at the thinner hinged portions. In some embodiments, after the microtips are bent out of plane to the desired angle, the substrate 110 is be cooled such that each of the microtips, now bent out of plane, are locked-in their upright orientations. In the instances where a substance, such as a vaccine, is already loaded on the microtips prior to bending out of co-planarity, localized heating of only the hinged regions of each microtip avoids thermal decomposition of the substance residing out toward the distal ends of the microtips.

With continued reference to FIG. 7, an excised, filled MicroArray 174 having the microtips bent such that they comprise projections emanating from the substrate 110 is depicted. In some embodiments, the bending of each microtip 100 out of the plane of the substrate 110 is accomplished by using a sandwich jig having protrusions corresponding to the location of each microtip on MicroArray sheet, such that when the sandwich jig is tightened together, these protrusions push against each microtip, bending each microtip out of planarity with the substrate. In some embodiments, differing configurations of jigs are employed to selectively bend only certain numbers of or certain regions of microtips selectively. For example, one jig is used to bend half the microtips to an angle of +80°, and then a second jig used to bend the other half of the microtips to an angle of 100°.

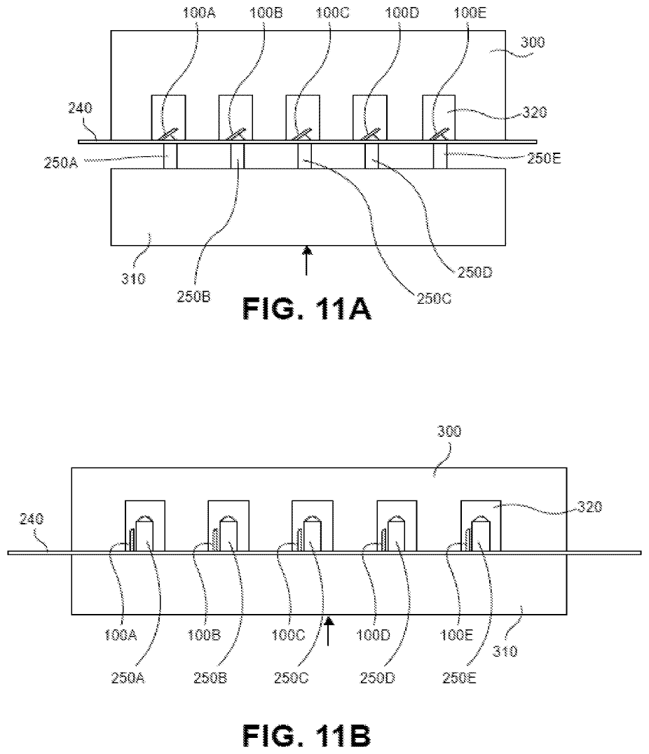

In some embodiments, in a substrate comprising rows and columns of photoetched MicroArrays, an entire row of MicroArray microtips are simultaneously bent into the Z-plane. For example, in some embodiments, a forming press apparatus is utilized to facilitate the bending of MicroArray microtips into the Z-plane; sec FIGS. 11A-B. In some embodiments, a forming press comprises a plurality of forming supports and forming dies that simultaneously bend an entire row of MicroArray microtips from the X/Y-plane of the MicroArray sheet 240 into the Z-plane. In some embodiments, each forming die 310 in the forming press comprises a plurality of projections including a first projection 250A, a second projection 250B, a third projection 250C, a fourth projection 250D, and a fourth projection 250E, for example, that facilitate the bending of a first microtip 100A, a second microtip 100B, a third microtip 100C, a fourth microtip 100D, and a fifth microtip 100E at the microtip hinge region into the Z-plane (e.g., FIGS. 11A-B). In some embodiments, each forming support 300 in the forming press comprises a clearance area 320 that allows the individual microtips to project into the Z-plane (e.g., FIGS. 11A-B). To produce Z-plane bent microtips, in some embodiments, the forming press apparatus presses the plurality of forming dies and forming supports together to cause the plurality of projections in each forming die to bend each microtip 100 up into the Z-plane of the clearance area 320 of the forming support 300 (e.g., FIG. 11B). Once the forming press is retracted, an entire row of MicroArray microtips are bent into the Z-plane.

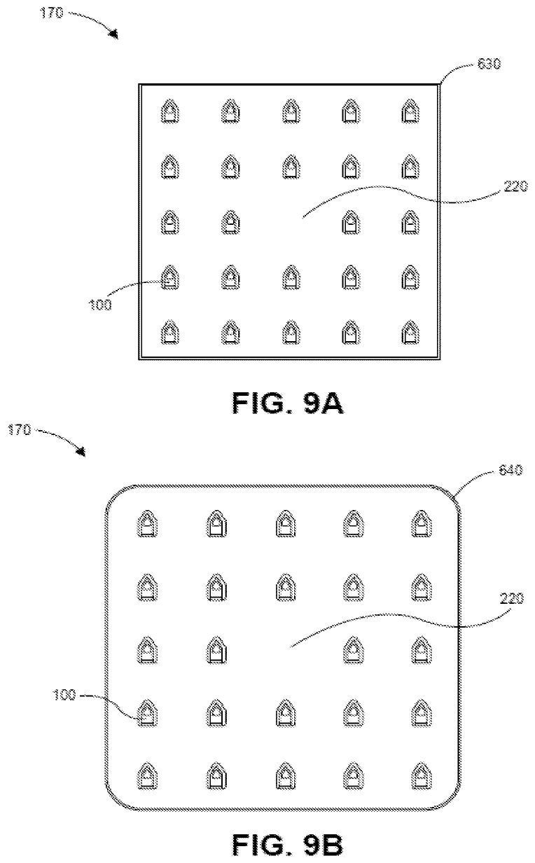

In some embodiments, a substrate comprising rows and columns of photoetched MicroArrays (e.g., FIG. 10), further comprises a fiducial marker 650, which facilitates the accurate location of individual MicroArrays within the rows (i.e., x-axis) and columns (i.e., y-axis) of the substrate. For example, in some embodiments, the forming press apparatus is operably linked to an SMT system (supra) that utilizes the spatial organization of the fiducial markers to align the forming press over a row of MicroArrays to bend the MicroArray microtips into the Z-plane. In some embodiments, the MicroArrays comprise a “pick-and-place” point 220, as shown in FIGS. 9A-9B. The pick-and-place point is the area on the top surface of the MicroArray that contains no microtips, where the SMT or P&P robotic arm picks-up the MicroArray without damaging the position and/or contents of the microtips.

It should be noted that in the instances where microtips are essentially two-dimensional, (absent reservoirs), microtips are coated with a substance after the microtips are bent into projections, although, as mentioned, this is likely to create waste of the substance that could be a precious drug substance. For example, in some embodiments, a substrate sheet is etched/die-cut, the microtips pushed out to about 90° using a sandwich jig or other suitable apparatus, and then the microtip projections coated with a substance by dipping, spraying or roll-coating. On the other hand, with the presence of a reservoir on each microtip, in some embodiments, the manufacturing steps are reversed and the inefficient, wasteful dip and roll-coating methods avoided. Thus, in some embodiments, of the disclosed methods of manufacturing a substance-loaded MicroArray, the substrate sheet is etched (e.g. photochemically), the reservoirs are etched (e.g. photochemical half etching) into the microtips, the reservoirs precisely filled with the substance, and then the microtips are bent out of planarity using the sandwich jig, forming press apparatus, or other suitable apparatus. Reversing the steps, and including reservoirs, allows for the replacement of dipping/roll-coating with precision dispending—a change that is vital for rare, precious drugs such as vaccines. In various embodiments, photochemical etching simultaneously creates the microtip outlines in the substrate and removes a portion of each microtip to create the reservoirs, streamlining the process and allowing for marked scale-up in volume/speed of production.

### Excising MicroArrays from the Substrate

In some embodiments, once MicroArray microtips are loaded with a substance and bent out of plane from the substrate, individual MicroArrays are excised from the substrate. In some embodiments, individual MicroArrays are excised from the substrate using a punch press. In some embodiments, in a substrate comprising rows and columns of photoetched MicroArrays, an entire row of MicroArray microtips are simultaneously excised from the substrate. For example, in some embodiments, a punch press apparatus comprising (1) a punch array comprising a plurality of punch dies and (2) a clamp array comprising a plurality of clamps is utilized to simultaneously excise an entire row of MicroArrays from the substrate. To produce excised MicroArrays, in some embodiments, the punch press apparatus presses the punch array and the clamp array together to cause the plurality of punch dies in the punch array to excise individual MicroArrays in a row of MicroArrays. Once the punch press is retracted, an entire row of individual MicroArrays have been excised from the substrate and are suitable for further processing.

### MicroArray Patch

In various embodiments, a MicroArray in accordance with the present disclosure is fixed to an adhesive and/or adsorbent pad or patch to make a complete medical device (“MicroArray Patch” or “MAP”). In various aspects, a MicroArray Patch comprises an adhesive or absorbent pad and at least one MicroArray. To form a MicroArray Patch in accordance with the present disclosure, a MicroArray is attached to an adhesive patch or adsorbent pad by any means, e.g. using an adhesive, thermal welding, ultrasonic welding, etc.

The combination of MicroArray and patch is illustrated in FIG. 8, which depicts an excised, filled MicroArray 174, (with filled reservoirs and with each microtip bent out of planarity to a desired angle), attached to an adhesive disc 160 to produce a transdermal patch 180. The adhesive disc 160 has any suitable shape, such as square, rectangular, triangular, round, oval, etc. as needed. There is also no limit to the thickness or composition of the adhesive disc 160 used in conjunction with a MicroArray to form a MicroArray Patch 180. In some embodiments, the adhesive disc 160 is an absorbent patch. In various embodiments, the MicroArray Patch also includes a substance within the adhesive or adsorbent pad to be delivered transdermally in combination with the substance loaded onto the microtips to be delivered into the dermis.

### MicroArray Delivery Device and Methods

In various applications, the MicroArrays and transdermal patches comprising a MicroArray are dispensed by a spring loaded delivery device. In some embodiments, a MicroArray or transdermal patch comprising a MicroArray is affixed to the end of a plunger. Such a device aids a user in affixing the array or patch firmly against the skin, ensuring that all of the microtips in the MicroArray are at the required depth in the skin. In some embodiments, the delivery device is driven by a spring, and in some cases the device is provided to a patient in a cocked and loaded configuration. The method of using the delivery device comprises holding the open end of the device (containing the MicroArray patch 180) against the skin and pressing a trigger to release the cocked plunger. This action causes the plunger to quickly lunge forward, driving the MicroArray patch 180 into the skin of the patient. The force at which the plunger embeds the MicroArray patch 180 into the patient is pre-adjusted by choosing different springs for the device, and/or changing the length at which the spring is compressed. Settings on the device are provided such that the manufacturer changes the degree of force at which the plunger will operate, and in some embodiments the patient will be prevented from doing so by a closure. In some embodiments, the patient then covers the embedded MicroArray patch 180 with a bandage, or the array thus delivered already has an adhesive patch or other covering over the array that the patient adjusts after affixing the array on the skin. In exemplary configurations, the delivery device 190 is configured to deliver both the MicroArray patch 180 and the adhesive in a single step, in order to simplify the administration and improve patient compliance.

In some embodiments, the MicroArrays and/or MicroArray patches are delivered to the patient manually by simply pressing the MicroArrays and/or the MicroArray patches directly against the patient's skin. In some embodiments, the MicroArrays and/or MicroArray patches are delivered to an arm of a patient. In some embodiments, the MicroArrays and/or MicroArray patches are delivered to a fingertip of a patient.

### MicroArray Manufacturing Processes

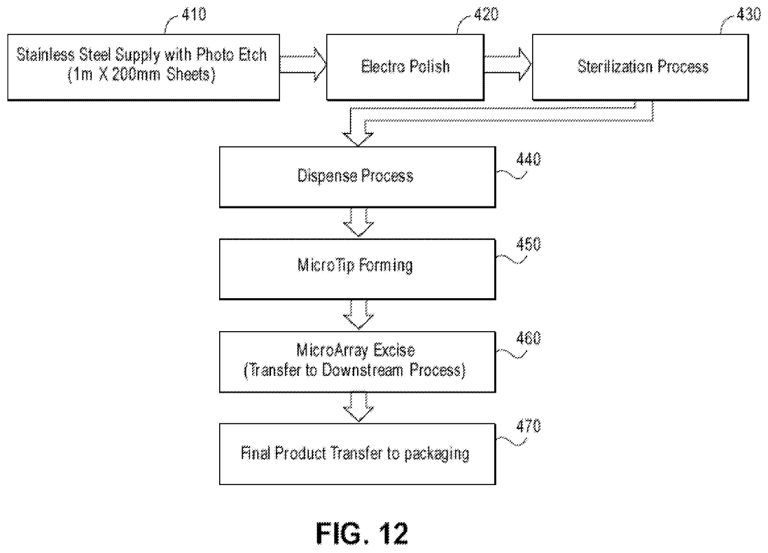

Referring now to FIG. 12, a MicroArray production process flow chart is shown. In some embodiments, the production process begins with the photoetching of a stainless steel sheet substrate, as shown by step 410. In some embodiments, the stainless steel sheets have a length of 1 m and a width of 200 mm. During step 410, the microtips are created by photo etching voids surrounding the microtip. Additionally, the hinged portions are created by photo etching the base of the microtips to a specific depth, as previously discussed. After the microtips and hinged portions have been created, the MicroArrays are electropolished to smooth, deburr, and/or streamline the microscopic surfaces of the microtips, as indicated by step 420. The MicroArrays are subsequently sterilized in step 430. In some embodiments, the MicroArrays are sterilized and/or depyrogenated by exposure to heat; exposure to gamma radiation; autoclaving; applying sodium hydroxide, muriatic acid, phosphoric acid, and/or nitric acid; cleaning with hot water and a detergent and subsequently spraying with a sterile solution; applying a non-polar solvent; applying a polar solvent; or any combinations thereof. Once the MicroArrays are sterilized, the substance (e.g. a vaccine) is dispensed into the reservoirs of the microtips, as shown in step 440. In some embodiments, dispensing of a substance into a reservoir of a microtip is done manually or automatically. In some embodiments, manually dispensing of a substance into the reservoirs comprises manually pipetting the substance into the reservoir. In some embodiments, automatically dispensing of a substance into the reservoirs comprises use of an automated, low volume microfluidic dispensing device, an automated liquid handler, a printer, or any other suitable automated low volume dispensing devices. Step 450 indicates the bending of the microtips into the Z-plane after the dispensed substance has dried. In some embodiments, the bending of the microtips is done manually or automatically. In some embodiments, the manual bending of the microtips comprises the use of a jig comprising projections that align directly beneath each microtip and bend each microtip into a Z-plane when force is manually applied, as previously discussed. In some embodiments, the automatic bending of the microtips comprises the use of an automated forming support further comprising a forming die with a plurality of projections that bend the microtips into a Z-plane when a force is automatically applied, as previously discussed. The MicroArrays are excised in step 460. In some embodiments, excision of the MicroArrays is done manually or automatically. In some embodiments, manual excision of the MicroArrays comprises the use of a die to cut each MicroArray, one by one, from the stainless steel sheet substrate. In some embodiments, automatic excision of the MicroArrays comprises the automated and simultaneous use of multiple dies (e.g. a row of five dies) to cut more than one MicroArray (e.g. a row of five MicroArrays) at a time. The excised MicroArrays comprising loaded, bent microtips are then transferred as a final product to be packaged, as indicated by step 470.

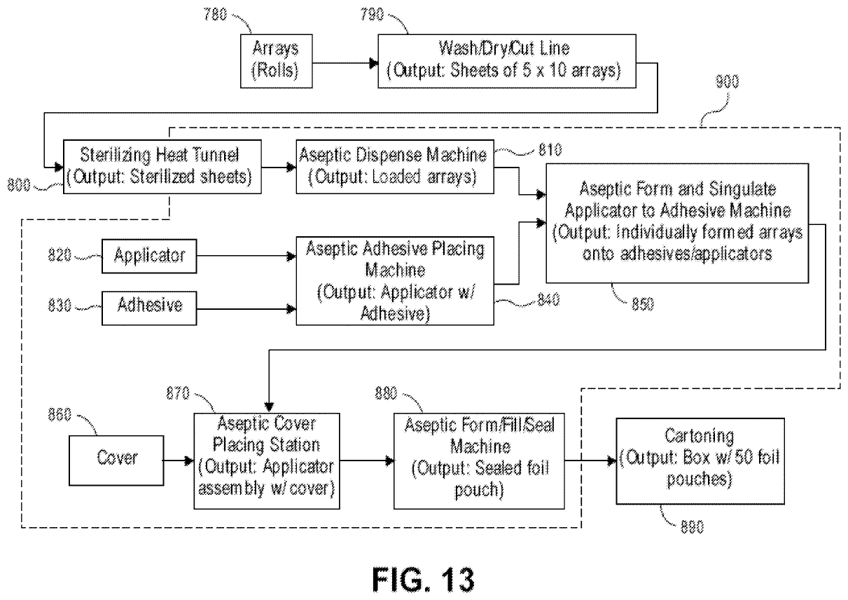

Referring now to FIG. 13, a manufacturing layout illustrating the various machines and their functions in the MicroArray patch manufacturing process is shown. Step 780 indicates the process begins with rolls of stainless steel sheets that are then washed, dried, and photoetched into MicroArrays, as indicated by step 790 (i.e. “arrays” in steps 780 and 790). At the end of step 790, a total of 50 MicroArrays are formed onto each stainless steel shect (e.g. in a 5×10 array). The MicroArrays, which are still attached to the stainless steel sheet at this point, are then sterilized in a sterilizing heat tunnel, as shown in step 800. In some embodiments, the MicroArrays are sterilized and/or depyrogenated by exposure to a temperature of up to 320 degrees for at least 30 minutes. As shown in step 810, an aseptic dispense machine aseptically dispenses a substance into the reservoirs of the microtips. Simultaneously and in parallel, an applicator 820 and an adhesive 830 are assembled in step 840. In step 850, the sterile, loaded MicroArrays from step 810 are excised and the excised MicroArrays are assembled with the applicator (i.e. a MicroArray delivery device) comprising an adhesive to form applicators (i.e. a MicroArray delivery devices) comprising MicroArray patches. A cover 860 is used to cover the applicators (i.e. a MicroArray delivery devices) comprising the MicroArray patches in an aseptic cover placing station in step 870. The aseptically covered applicators or MicroArray delivery devices comprising the MicroArray patches are then placed in a sterile foil pouch that is aseptically sealed with the aseptic form/fill/seal machine in step 880. The final product is then sent to a cartoning machine to be packaged, as indicated by step 890. In some embodiments, the cartoning machine picks up a folded carton, erects it, fills it with the sterile foil pouch containing the aseptically covered applicator comprising the MicroArray patch through an open end, and closes the carton by either tucking the flaps of the carton or by applying an adhesive, thereby packaging the final product. Dotted line 900 indicates an aseptic barrier; in other words, all the steps enclosed within dotted line 900 are to be performed under aseptic conditions. Similarly, all machines used to perform the steps enclosed within dotted line 900 are to be kept in aseptic conditions. Best practices for restricted access barrier systems in aseptic manufacturing are well known in the art and include, but are not limited to, high level disinfection, the use of sterile gloves, aseptic transfer systems for zone transitions (e.g. transition from an ISO 5 area to an ISO 4 area), and closed door policy (e.g. during filling).

### Packaging and Kits Including Same

MicroArrays are provided in some type of packaging depending on whether a substance is already on the microtips, the nature of that substance on the microtips, and the nature of the user receiving the array (e.g. intermediary third party versus end use patient), amongst other considerations. Etched MicroArrays, for example not yet completed in some regard, such as not loaded with a substance, are packaged in bulk cartons for shipment to the appropriate third party to complete. In some embodiments, such packaging is typical corrugate, with the necessary padding and separation layers (e.g. tissue paper or cloth) to protect the etched substrate material. In some embodiments, desiccants (e.g. silica gel packets) and/or antioxidant materials are added inside the cartons to mitigate oxidation to the metal substrate.

In some embodiments, for MicroArrays loaded with temperature, moisture, and/or air sensitive substances, the packaging for the MicroArray is much more sophisticated than a simple carton. In various aspects, microtip packaging comprises a laminated pouch providing at least one of oxygen and moisture barrier properties. In some embodiments, pouches for protecting substance loaded microtips have any number of lamination layers and types of materials as necessary for the desired barrier properties. For example, a pouch comprises any number and combination of foil and plastic film layers such that the desired level of oxygen and/or moisture blockage is achieved. In some instances, laminated pouches provide some degree of thermal protection, although temperature control is also accomplished by placing one or more laminated pouches inside a Styrofoam cooler containing ice or dry ice.

In various embodiments, a MicroArray is loaded with a vaccine further comprising RNA. In some embodiments, such MicroArrays are stabilized with RNAstable or other RNA stabilizing agents, and then also packaged in a laminated pouch for further protection against degradation of the vaccine. A laminated pouch useful for protecting MicroArrays having RNA include 3-layer laminated pouches comprising polyethylene terephthalate/foil/cast polypropylene layers (abbreviated as “PET/foil/CPP”). A representative 3-layer pouch is available from Ampac under the brand name FoilPAK® KSP-150. In some embodiments, any shape of pouch is used for packaging a MicroArray in accordance with the present disclosure, such as a gusseted pouch or other type. When a substance loaded MicroArray is packaged in such a laminated pouch, the packaging process is performed under controlled conditions, such as to exclude oxygen and moisture from inside the package. For example, a substance loaded MicroArray is packaged in a pouch such as the PET/foil/CPP pouch under dry nitrogen or argon conditions, or under vacuum, to exclude air and moisture from the pouch. In some embodiments, a desiccant packet and/or an oxygen-absorbing packet is also be inserted into the pouch along with the substance loaded MicroArray under the inert atmosphere conditions. In some embodiments, the filled pouches are then be heat sealed, glued, or bonded in any other way to protect the substance loaded MicroArray.

In various embodiments, the procedure for filling barrier pouches with a MicroArray comprises the automated packaging process known as “form-fill-seal.” Machines are available to make the pouches from the appropriate films, fill the pouches, and then seal the filled pouches. Automated equipment capable of form-fill-seal is available from Multivac and other suppliers. In some embodiments, film material is found at Bemis Company, Inc. In some embodiments, separate heat sealing equipment is employed, such as an Accu-Seal Model 8000 heat scaling machine.

The number of MicroArrays packaged within each pouch depends on the nature of the receiving party and the intended use. For example, a single substance loaded MicroArray in a protective pouch is provided to a pharmacy, a clinic, or directly to a patient. In other examples, several MicroArrays are packaged in a single protective pouch, such as to a single patient to be used in accordance to a prescription, or supplied to a clinic for dispensing to several patients. In some embodiments, if an additional manufacturing step is still necessary, several MicroArrays are placed and shipped to a third party manufacturer in a protective pouch.

In accordance to the present disclosure, a microtip kit comprises at least one MicroArray and a package containing the MicroArray. In some embodiments, as mentioned above, the MicroArray is loaded with a substance, such as a vaccine, and the packaging comprises a protective pouch further comprising any number and combination of laminated layers. In some embodiments, the protective pouch provides a barrier to at least one of temperature extremes, oxygen and moisture. In various embodiments, the kit further comprises at least one of a desiccant packet, an oxygen-absorbing packet, and an instruction sheet or label. In certain aspects, the label of the kit is affixed to the exterior surface of the protective pouch, and the desiccant packet and/or oxygen-absorbing packet placed inside the protective pouch along with the MicroArray. In some embodiments, an FDA drug label, for example, is glued on the outside of the pouch, folded as necessary to fit to the size of the panel available for labeling. In various embodiments, the kit also includes instruction sheets or booklets, provided outside the protective pouches, such as in secondary packaging used to hold one or more pouches and an instruction booklet. For example, a kit shipped to a pharmacy includes a single exterior corrugated carton or insulating cooler containing an instruction booklet teaching how to dispense and use the MicroArrays, ice or dry ice, and several or more individual protective pouches each containing a MicroArray (e.g. loaded with a sensitive vaccine) inside and a label (e.g. an FDA drug label) affixed to the outside. In some embodiments, within each pouch of the kit, there is an inert gas or a vacuum, a desiccant packet, and/or an oxygen-absorbing packet.

### Exemplary Embodiments of the Present Disclosure

In various embodiments, the present disclosure provides MicroArrays and novel methods for making same, and in various aspects, provides MicroArrays prepared by a process of etching or cutting and bending portions of substrate sheets to form microtip projections:

In various embodiments, a method of manufacturing a MicroArray comprises the steps of: (i) providing a substrate; (ii) photochemically etching a plurality of microtips in said substrate; (iii) configuring a reservoir into each microtip; (iv) dispensing an amount of a substance into each reservoir; and (v) bending each microtip out of planarity to an angle relative to the plane of the substrate. In various examples, the step of configuring a reservoir comprises photochemical etching. In certain aspects, the step of configuring a reservoir comprises photochemical half etching of some of the thickness of the substrate material. In some embodiments, steps (ii) and (iii) occur simultaneously in a photochemical etching process.

In various embodiments, a method of manufacturing a MicroArray comprises the steps of: (i) providing a substrate; (ii) cutting a plurality of microtips in said substrate; (iii) configuring a reservoir into each microtip; (iv) dispensing an amount of a substance into each reservoir; and (v) bending each microtip out of planarity to an angle relative to the plane of the substrate. In some examples, both the steps of configuring the reservoirs and filling each are optional steps, and instead, the method comprises a step of coating generally two-dimensional microtips with a substance after the step of bending each out of planarity. In various embodiments, the microtip projections are coated with a substance such as a drug composition or vaccine in a dipping or roll-coating or other suitable coating operation.

In some embodiments, the step of configuring a reservoir in a microtip comprises the denting of the substrate at each microtip using a punch or other suitable tool. In some embodiments, for metal substrates, a punch is used to dent the metal substrate to form each reservoir. In some embodiments, in the case of plastic substrates, a heated punch is used to partially melt/soften a portion of each microtip and to stretch out those softened portions to create depressions.

In some embodiments, the step of cutting a plurality of microtips into a substrate and the step of configuring a reservoir into each microtip occurs simultaneously, as mentioned such as through photochemical etching, or such as by providing and using an appropriately configured tools that comprises a combination of sharp die-cutting projections and rounded-end punch projections. In some embodiments, one punch with such an appropriate configured tool die-cuts and dents portions of the substrate sheet to form an array. In certain variations, the step of cutting a plurality of microtips into a substrate comprises laser ablation. As discussed, in some embodiments, photochemical etching, electrochemical etching, die-cutting, laser-cutting, waterjet cutting, and/or laser ablation are used in any combination, and the steps (etching, cutting, ablating, and optionally denting-out reservoir depressions) are conducted in any order whatsoever, and steps combined as desired. In various embodiments, proximal hinged portions of microtips are engineered to thicknesses that facilitate bending of the microtips across their hinged portions. In some embodiments, the thinning of hinged portions of the microtips is provided by photochemical etching and/or laser ablation.

In cases where a reservoir is desired that is not bordered around its entire periphery with walls, the step of configuring a reservoir into each microtip comprises photochemical etching and/or laser ablation of a portion of the thickness of the substrate at each microtip. Photochemical etching and laser ablation, for example, are used to produce the change in thickness observed in the microtip in FIG. 1, and in exemplary cases, are used to remove up to about 80% of the thickness of the substrate. In some embodiments, photochemical etching and/or laser ablation is also used to create microtip hinges wherein up to about 80% of the thickness of the substrate material is removed to create each hinged portion of each microtip.

In some embodiments, in any MicroArray, the microtips comprise a microtip density of from about 1 to about 1,000 microtips per square centimeter of substrate. For example, a suitable density ranges from about 10 to about 100 microtips per square centimeter of substrate area, or about 25 microtips per square centimeter.

In some embodiments, in various production methods, a substance is loaded into each reservoir of each microtip in an amount of from about 0.1 to about 5 nL, from about 1 nL to about 2 nL, from about 1 nL to about 10 nL, from about 2 nL to about 3 nL, from about 3 nL to about 4 nL, from about 4 nL to about 5 nL, from about 5 nL to about 6 nL, from about 6 nL to about 7 nL, from about 7 nL to about 8 nL, from about 8 nL to about 9 nL, from about 9 nL to about 10 nL, from about 10 nL to about 15 nL, from about 15 nL to about 20 nL, from about 20nL to about 25 nL, from about 25 nL to about 30 nL, from about 30 nL to about 35 nL, or from about 35 nL to about 40 nL of a fluidic material, such as by using an automated dispenser. In various aspects, a step of drying this substance after the step of dispensing an amount of the substance into each reservoir is added to the method. In some embodiments, dry substances are produced by drying liquid substances, or are powder coated or sublimed and condensed directly onto microtips. In some embodiments, each microtip comprises, for example, from about 0.2 ng to about 5 μg of dry substance.

In some embodiments, photochemical etching, die-cutting and/or laser-ablation of a substrate sheet are conducted such that each microtip appears as an outline, with salient features such as a reservoir, yet remaining co-planar with the substrate sheet. In some embodiments, the portion of the microtip not fully etched/cut or ablated away from the substrate is used as a hingeable region for bending each microtip out of planarity such that each becomes a projection emanating from the substrate. In various embodiments, each microtip comprises a hingeable portion at the proximal end and a sharp distal end. In some embodiments, hingeable regions are temporarily softened by heating to facilitate bending without distortion of shape or features. In other aspects, a thinner hinging region eliminates any need for localized heating to facilitate bending.

In various embodiments of the present disclosure, a MicroArray comprises: a substantially planar substrate further comprising a plurality of drug loaded microtip projections, each of said microtip projections projecting at an angle of about 90° relative to the substantially planar substrate. In some embodiments, each of the microtip projections are hingeably attached to the substrate, and each further comprises a depression into which a substance, such as a drug, is be loaded.

In various embodiments, as shown in FIG. 9A, a MicroArray comprises sharp corners 630. In some embodiments, as shown in FIG. 9AB, a MicroArray comprises rounded corners 640. Including rounded corners as part of the MicroArray design reduces the risk of injury (i.e. cuts, puncture wounds, etc.) to the patient being administered the MicroArray or MicroArray patch. In addition, a MicroArray design comprising rounded corners facilitates sterile packaging and scaling of the MicroArrays and/or MicroArray patches by preventing sharp corners from tearing the packages and/or pouches containing the MicroArrays and/or MicroArray patches.

In various embodiments, a MicroArray comprises: a substantially planar substrate further comprising a plurality of substance-loaded microtip projections, wherein each of the microtip projections project out at an angle relative to the substantially planar substrate, with the array formed by a process comprising: (i) providing the substrate; (ii) cutting a plurality of microtips in said substrate; (iii) configuring a reservoir into each microtip; (iv) loading an amount of a substance into each reservoir; and (v) bending each microtip out of planarity to an relative to the plane of the substrate to create each microtip projection. In some instances, the step of configuring a reservoir comprises the denting of the substrate at each microtip using a punch or other appropriately shaped tool with the proper imprint. In other instances, steps (ii) and (iii) comprise photochemical etchings, and steps (ii) and (iii) occurs simultaneously or sequentially in either order. In various embodiments, the method further comprises electropolishing of the microtips prior to step (iv). In some embodiments, optional addition of a lubricant also occurs prior to step (iv), just after the electropolishing as mentioned above.

It will be apparent to those skilled in the art that various modifications and variations are possible without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the appliance and/or methods. The disclosure is intended as illustrative only and as such is not intended to be exhaustive. It will be evident to those skilled in the art that various modifications are made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

In some embodiments, microtips and MicroArrays are designed for use with microfluidic dispensing devices. In some embodiments, microtips are designed and manufactured to have a center vacuum pen pick-up point or a “pick-and-place” point. In some embodiments, the pick-and-place point 220 is located in the center of the excised, empty, flat MicroArray 170, as shown in FIGS. 9A-B. In some embodiments, the pick-and-place point 220 is located in the center of the MicroArray and one row of the MicroArray contains at least one fewer microtip 100 compared to the remaining rows in the array. For example, a MicroArray in a 5×5 configuration will contain 25 total individual microtips. A similar 5×5 MicroArray with a pick-and-place point 220 located in the center of the MicroArray in place of a microtip will result in an array with 24 total microtips (see, e.g., FIGS. 9A-B). As used herein, a configuration of microtips in a MicroArray in which a microtip has been replaced by a pick-and-place point will be referred to as an “N×N−1” array (e.g., “5×5−1” or “3×3−1”). Likewise, a configuration of microtips in an array which two microtips have been replaced by a pick-and-place point will be referred to as an “N×N−2” array. In some embodiments, any suitable number of microtips (e.g., “N×N−3,” “N×N−4,” etc.) are replaced to facilitate the positioning of the pick-and-place point or any other feature. Any configuration of rows and columns of individual microtips in a MicroArray are contemplated with the methods and arrays disclosed herein. Similarly, in some embodiments, any number of microtips in a standard “N×N” array in any number of rows and/or columns are replaced by features such as a pick-and-place point. In some embodiments, the pick-and-place point is 5 mm by 5 mm. In some embodiments, the pick-and-place point is located at the exact center of the MicroArray. In some embodiments, the pick-and-place point is located at a top right corner, a top left corner, a bottom left corner, or a bottom right corner of the MicroArray. In some embodiments, the MicroArray comprises 2, 3, 4, or 5 pick-and-place points.

In some embodiments, a 5×5 MicroArray, as shown in FIGS. 9A-B, has a length of 10 mm and a width of 10 mm. In some embodiments, each microtip 100 of a 5×5 MicroArray, as shown in FIGS. 9A-B, is 2 mm away from an adjacent microtip 100. In some embodiments, each microtip 100 next to the edge of a 5×5 MicroArray, as shown in FIGS. 9A-B, is 0.75 mm away from the edge of the 5×5 MicroArray.

In some embodiments, microtips are designed and manufactured to have a “quick cut-out” design. A “quick cut-out” design facilitates isolation of individual manufactured MicroArrays from a larger manufacturing substrate (such as a sheet of MicroArrays). A “quick cut-out” design comprises the removal of a substantial portion of the MicroArray manufacturing substrate surrounding an individual MicroArray such that the array itself is only in contact with the substrate at one or more small contact points located on the periphery of an individual MicroArray. In some embodiments, a quick cut-out design creates individual MicroArrays that have four attachment points with a manufacturing substrate comprising multiple MicroArrays (see, e.g., FIG. 10). Any suitable quick cut-out design that facilitates case of isolation of MicroArrays from a manufacturing substrate is contemplated for use with the methods and arrays disclosed herein.

In some embodiments, the microtip is beveled (see, e.g., FIGS. 4A-I). Any suitable beveling orientation and patterning that: (1) decreases penetration force required for microtip application; (2) increases case of microtip application; or (3) reduces any pain or discomfort are contemplated for use with the microtips and manufacturing methods disclosed herein.

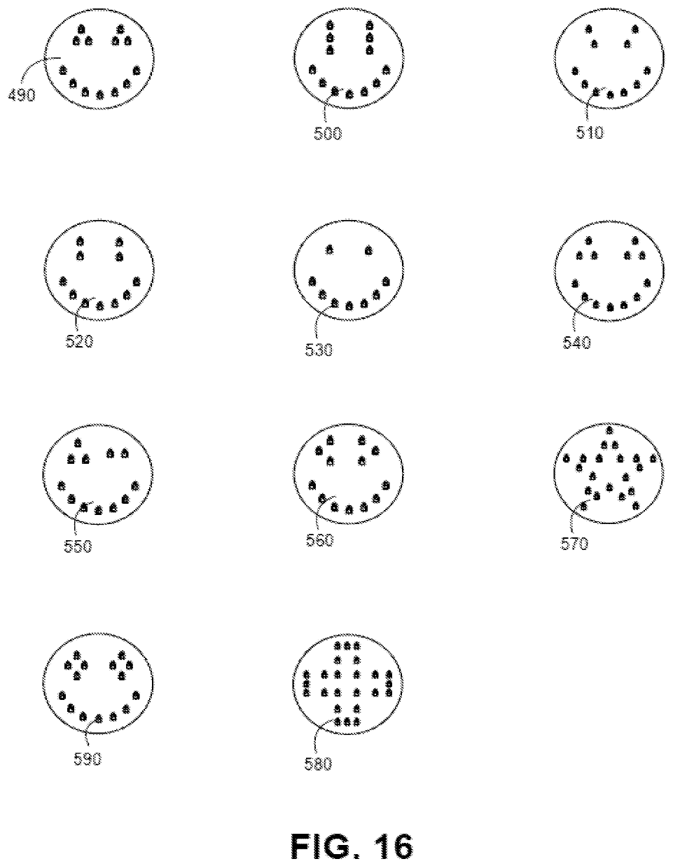

In some embodiments, the MicroArrays comprise a custom microtip design. FIG. 16 shows exemplary MicroArray customized designs that are utilized with the manufacturing methods disclosed herein. In some embodiments, the microtip design is a smiley face; different variations of smiley face designs are shown in FIG. 16 (see, e.g. 490, 500, 510, 520, 530, 540, 550, 560, and 590). In some embodiments, the microtip design is a cross 580 or a star 570. In some embodiments, a microtip design is one or more cartoon characters, one or more letters, one or more numbers, one or more geometrical shapes, or a combination thereof. In some embodiments, a custom microtip design is utilized to identify a substance that is administered to an individual. In some embodiments, the substance administered to an individual is a vaccine. In some embodiments, the custom microtip design is recognized by a computing device.

### Example 1—Manufacture of MicroArrays

Microtips and MicroArrays are prepared by photochemical etching of 3 mil (75 um) thick stainless steel 304 foil. After etching, individual 1 cm² MicroArrays containing a 5×5−1 array of individual microtips are loaded with a drug of interest (e.g., a vaccine) by a multi-channel microfluidic dispenser (e.g., a BioDot microfluidic printer). All 24 individual microtips in a two dimensional X, Y plane are loaded with 5-10 nL/needle in about 10 seconds. Once the microtips have been loaded and dried, the needles are placed in a sandwich jig to bend microtips to project outwards into the Z plane. The jig contains pegs that correspond to the 5×5−1 array such that when the sandwich is compressed the microtips are bent into the Z plane.

### Example 2—Sugar Glass Formulated MicroArrays

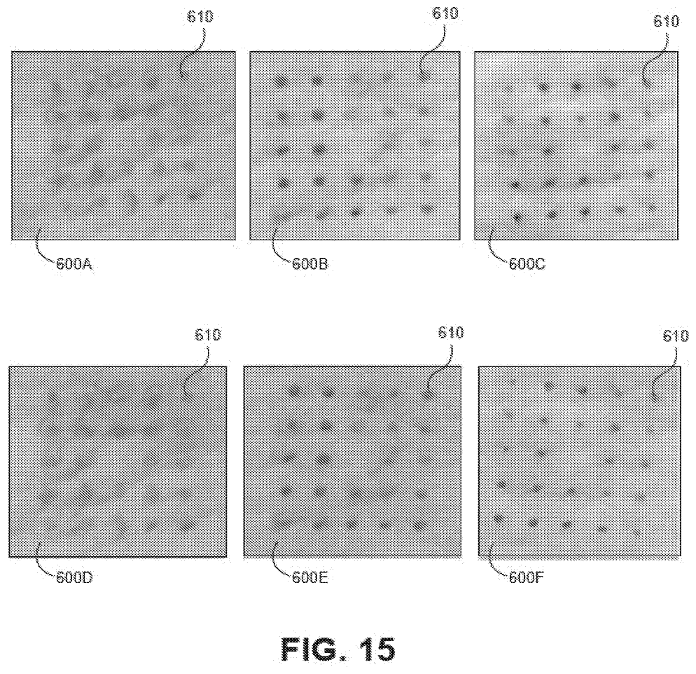

To test the solubility of dried trehalose in skin, a 30% trehalose/0.2% Congo Red-mix were microfluidically printed (i.e. dispensed by an automated microfluidic dispenser) on 5×5−1 MicroArrays (i.e., 24 total reservoirs per MicroArray). MicroArrays were dried at room temperature for 24 hours in a sealed box containing desiccant material. Frozen pig skin (pork belly) was thawed at room temperature, wiped dry with a tissue, and warmed to 37° C. (body temperature). Pre-warmed skin was wiped with 10% glycerol and, as shown in FIG. 15, MicroArrays were applied to the skin for 1 minute 600A, 5 minutes 600B, or 20 minutes 600C at 37° C. After the MicroArrays were removed, the application site was wiped with PBS to test if the trehalose/Congo Red mix 610 was located superficially on the skin and could be wiped away or if the trehalose/Congo Red mix was in the dermis.

MicroArray microtips for the 1 minute, 5 minute, and 20 minute applications contained no residual trehalose/Congo Red sugar glass sample indicating that the entire sugar glass sample had been transferred onto the skin. As seen in FIG. 15 (600D-F), the trehalose/Congo Red mix was clearly present in the deeper layers of the skin after a PBS wipe in MicroArrays applied for at least 5 minutes. 600D corresponds to the 1 minute MicroArray application after it was wiped with PBS. 600E corresponds to the 5 minute MicroArray application after it was wiped with PBS. 600F corresponds to the 20 minute MicroArray application after it was wiped with PBS. These results indicate that trehalose-stabilized MicroArrays effectively deliver a sample of interest into the deeper layers of the skin.

### Example 3—Microtip Arrangements

In accordance with the MicroArray manufacturing methods disclosed herein, MicroArrays and MicroArray Patches are produced in a variety of different shapes and sizes. Furthermore, in accordance with the manufacturing methods disclosed herein, in some embodiments, microtips on individual MicroArrays and MicroArray Patches are arranged into any number of configurations (e.g., FIG. 16). In addition to their aesthetically pleasing properties, configurations of microtips as exemplified in FIG. 16 are useful to encourage drug compliance (e.g., vaccine compliance).

### Example 4—Hepatitis B Vaccine-Loaded MicroArray Patch In Vivo Study

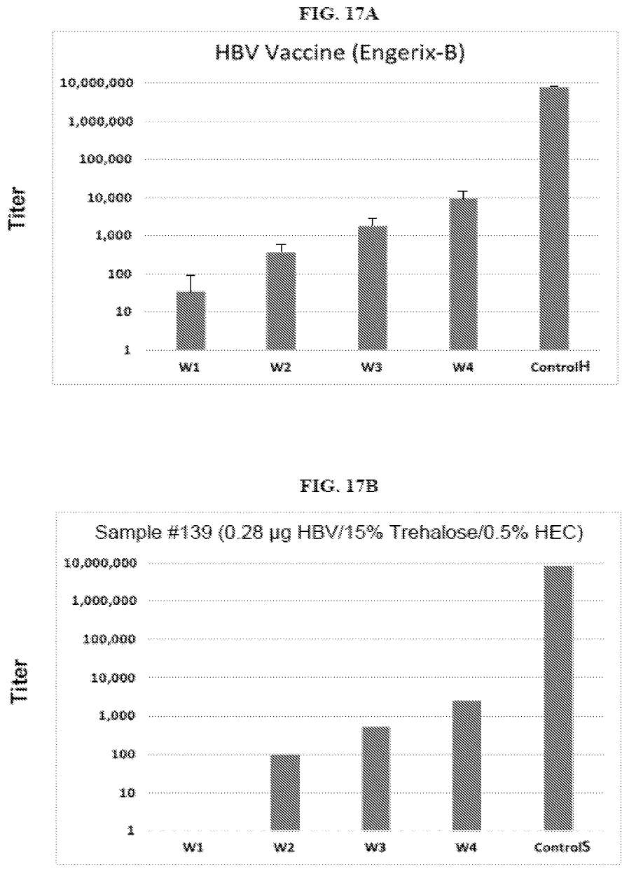

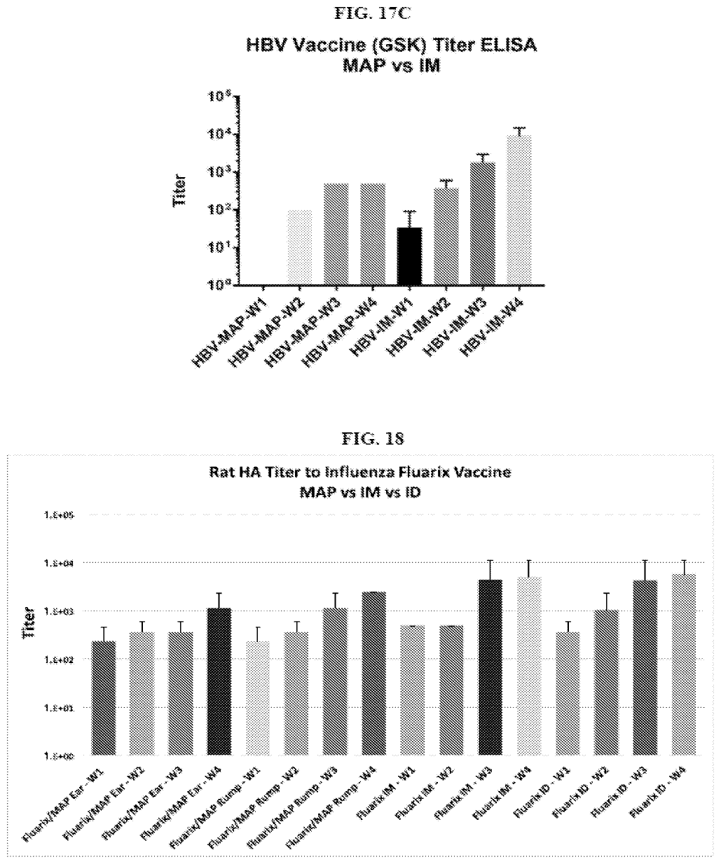

An in vivo, murine study was conducted to compare serum titer values induced by administration of a hepatitis B virus (HBV) vaccine via loaded MicroArray Patches vs. a conventional intramuscular (IM) administration, as shown in FIGS. 17A-C. The HBV vaccines were prepared as follows: HBV vaccine (Engerix-B®, GSK) was concentrated from a fresh (never frozen) vaccine. The vaccine was concentrated using Amicon-0.5 concentrators to 560 μg/ml (approximately 28-fold). Amicon concentrators are ultra-centrifugal filters designed for protein purification and concentration. The concentrated vaccine was mixed 1:1 with either nuclease-free water or a 30% Trehalose/1% Hydroxyethyl cellulose (HEC)-mix to yield a final HBV concentration of 280 μg/ml. The vaccine (0.28 μg total) was dispensed into reservoirs of microtips on 25% polished 5×5−1 (large) MicroArrays using a microfluidic dispenser. MicroArrays were attached to an adhesive disc to produce MicroArray Patches. MicroArray Patches were sealed in slide mailers and foil bags with four H₂O-scavenger sachets per foil bag and dried at room temperature (20° C.).

A first group of mice received an intramuscular (IM) injection of the HBV vaccine, as previously described. A second group of mice was administered the HBV vaccine via MicroArray Patches prepared as previously described. Mouse sera was collected and analyzed one, two, three, and four weeks post-immunization.

Total titer values after administration of the HBV vaccines were analyzed by enzyme-linked immunosorbent assays (ELISA). The ELISA tests were conducted as follows: a plate was coated overnight at 4° C. with HBsAg protein (the surface antigen of the HBV) at 0.5 μg/ml. The plate was washed three times with TBST (tris-buffered saline and Tween 20) and blocked with 5% BSA (bovine serum albumin) in tris-buffered saline (TBS) for 1 hour at room temperature. After washing, mouse sera (1:100-1:12500) and positive control (1:500-1:62500; horse polyclonal anti-HBsAg-antibody, Abcam) in 1% BSA/TBST were added and incubated for 2 hours at room temperature, followed by washing. Anti-mouse-SA antibody (sera) or anti-horse-SA antibody (positive control), 1:5000 in 1% BSA/TBST, was added and incubated for 1 hour at room temperature. The plate was washed again and then incubated with anti-SA (1:200) in 1% BSA/TBST for 20 min at room temperature. After washing, substrate was added and incubated for 30 minutes at room temperature. The reaction was stopped by addition of 50 μl 2N sulfuric acid.

FIG. 17A shows mouse titer values in mouse sera, one week (“W1”), two weeks (“W2”), three weeks (“W3”), and four weeks (“W4”) after a single IM administration of the HBV vaccine. A positive control (“Control”) was also included in the ELISA tests, as previously mentioned. FIG. 17B shows mouse titer values in mouse sera, two weeks (“W2”), three weeks (“W3”), and four weeks (“W4”) after a single dose of the HBV vaccine was administered via a MicroArray Patch. A positive control (“Control”) was also included in this study. FIG. 17C summarizes the data presented in FIGS. 17A-B. For example, HBV-MAP-W2 refers to the mouse sera titer detected 2 weeks after administration of the HBV vaccine via a MicroArray Patch (MAP); HBV-MAP-W3 refers to the mouse sera titer detected 3 weeks after administration of the HBV vaccine via a MicroArray Patch (MAP); HBV-MAP-W4 refers to the mouse sera titer detected 4 weeks after administration of the HBV vaccine via a MicroArray Patch (MAP); HBV-IM-W1 refers to the mouse sera titer detected 1 week after intramuscular (IM) administration of the HBV vaccine; HBV-IM-W2 refers to the mouse sera titer detected 2 weeks after intramuscular (IM) administration of the HBV vaccine; HBV-IM-W3 refers to the mouse sera titer detected 3 weeks after intramuscular (IM) administration of the HBV vaccine; and HBV-IM-W4 refers to the mouse sera titer detected 4 weeks after intramuscular (IM) administration of the HBV vaccine. Standard deviation is shown on the IM conditions.

Anti-HBV IgG antibodies at a titer of 1:100-1:12,500 were detected for mouse sera #141-143 (IM injected HBV vaccine). Sample #139 (0.28 μg HBV/15% Trehalose/0.5%HEC loaded onto MicroArray Patches) had a positive titer of 1:500 at weeks 3 and 4 and a titer of 1:100 at week 2. Hence, these results indicate that the transdermal delivery of an HBV vaccine using the MicroArray Patches disclosed herein induced a strong immune response as efficiently as the standard intramuscular immunization.

### Example 5—Influenza Virus Vaccine-Loaded MicroArray Patch In Vivo Study

An in vivo, rat study was conducted to compare serum titer values induced by administration of an influenza virus vaccine via loaded MicroArray Patches vs. an intradermal (ID) administration and vs. a conventional intramuscular (IM) administration, as shown in FIG. 18. The influenza vaccines were prepared as follows: the influenza vaccine was concentrated from fresh (never frozen) GSK Fluarix® quadrivalent influenza vaccine (approximately 20-fold). The vaccine was concentrated using Amicon-0.5 concentrators (i.e. ultra-centrifugal filters designed for protein purification and concentration) to 600 μg/ml per hemagglutinin (HA). The concentrated vaccine was then mixed 1:1 with 30% Trehalose with 0.4% Congo Red to yield a final concentration of 300 ug/ml per HA and 15% Trehalose. The vaccine (0.3 μg total) was dispensed with a microfluidic dispenser (8×5 nL, i.e. a total of 40 nL) into the reservoirs of 25% polished 5×5−1 (large) MicroArrays. Six loaded MicroArrays were stored at room temperature to allow the vaccine to dry within the reservoirs of the microtips. After the vaccine dried, MicroArrays were attached to an adhesive disc to produce MicroArray Patches. MicroArray patches were sealed in slide mailers and foil bags with four H₂O-scavenger sachets per foil bag.

A first group of rats (n=3 animals) received an intradermal (ID) injection of 0.3 μg influenza virus vaccine in 50 μl sterile PBS (Phosphate Buffered Saline). A second group of rats (n=6 animals) was administered the influenza virus vaccine via MicroArray Patches prepared as previously described. A third group of rats (n=3 animals) received an intramuscular (IM) injection of 1.5 μg influenza virus vaccine in 50 μl sterile PBS. For FIG. 18, rat sera was collected and analyzed one, two, three, and four weeks post-immunization.

Total titer values after administration of the influenza vaccines were analyzed by enzyme-linked immunosorbent assays (ELISA). The ELISA tests were conducted as follows: a plate was coated overnight at 4° C. with HA protein at 0.5 μg/ml. The plate was washed three times with TBST (tris-buffered saline, 0.05% Tween 20) and blocked with 5% BSA (bovine serum albumin) in TBS (tris-buffered saline) for 1 hour at room temperature. After washing, rat sera (1:100-1:12500) and a positive control (1:62,500-1:7,812,500; monoclonal anti-HA-antibody, ImmuneTech) in 1% BSA/TBST were added and incubated for 2 hours at room temperature, followed by washing. Anti-mouse-HRP (Horseradish peroxidase) antibody, 1:5000 in 1% BSA/TBST, was added and incubated for 1 hour at room temperature. The plate was washed again and then incubated with substrate for 30 minutes at room temperature. The reaction was stopped by addition of 50 μl 2N sulfuric acid.

Rat sera were diluted 5-fold (1:500-1:12500). The titer is defined as the reciprocal of the highest sample dilution that gives an absorbance reading above the cutoff. The cutoff is defined as the absorbance that is two times higher than the mean background.

FIG. 18 compares an intradermal (ID) influenza virus immunization vs. an influenza virus vaccine administered via a MicroArray Patch (MAP) vs. an intramuscular (IM) influenza virus immunization in rats. For example, Fluarix/MAP Ear—W1 refers to the rat sera titer detected 1 week after administration of the influenza virus vaccine via a MicroArray Patch (MAP) on the rat's ear; Fluarix/MAP Ear—W2 refers to the rat sera titer detected 2 weeks after administration of the influenza virus vaccine via a MicroArray Patch (MAP) on the rat's ear; Fluarix/MAP Ear—W3 refers to the rat sera titer detected 3 weeks after administration of the influenza virus vaccine via a MicroArray Patch (MAP) on the rat's ear; Fluarix/MAP Ear—W4 refers to the rat sera titer detected 4 weeks after administration of the influenza virus vaccine via a MicroArray Patch (MAP) on the rat's ear.

In addition, Fluarix/MAP Rump—W1 refers to the rat sera titer detected 1 week after administration of the influenza virus vaccine via a MicroArray Patch (MAP) on the rat's rump; Fluarix/MAP Rump—W2 refers to the rat sera titer detected 2 weeks after administration of the influenza virus vaccine via a MicroArray Patch (MAP) on the rat's rump; Fluarix/MAP Rump—W3 refers to the rat sera titer detected 3 weeks after administration of the influenza virus vaccine via a MicroArray Patch (MAP) on the rat's rump; Fluarix/MAP Rump—W4 refers to the rat sera titer detected 4 weeks after administration of the influenza virus vaccine via a MicroArray Patch (MAP) on the rat's rump.

Furthermore, Fluarix IM—W1 refers to the rat sera titer detected 1 week after intramuscular (IM) administration of the influenza virus vaccine; Fluarix IM—W2 refers to the rat sera titer detected 2 weeks after IM administration of the influenza virus vaccine; Fluarix IM—W3 refers to the rat sera titer detected 3 weeks after IM administration of the influenza virus vaccine; Fluarix IM—W4 refers to the rat sera titer detected 4 weeks after IM administration of the influenza virus vaccine.

Lastly, Fluarix ID—W1 refers to the rat sera titer detected 1 week after intradermal (ID) administration of the influenza virus vaccine; Fluarix ID—W2 refers to the mouse sera titer detected 2 weeks after ID administration of the influenza virus vaccine; Fluarix ID—W3 refers to the mouse sera titer detected 3 weeks after ID administration of the influenza virus vaccine; Fluarix ID—W4 refers to the mouse sera titer detected 4 weeks after ID administration of the influenza virus vaccine.

As shown in FIG. 18, anti-HA IgG antibodies at a titer of 1:500-1:12500 were detected in rat sera of the rats that were administered the influenza virus vaccine via the MicroArray Patches on both the car and the rump. In addition, anti-HA IgG antibodies at a titer of 1:2500 were detected in rat sera of the rats that were administered the influenza virus vaccine via both the IM and ID injections. Rats that received the influenza virus vaccine via an IM injection and via a MicroArray Patch had similar titer values of anti-HA IgG antibodies as the titer values produced by the IM and ID injections. The addition of Congo Red did not pose a problem for this particular influenza virus vaccine (Fluarix) as titers for MAPs applied to rump and car are only slightly lower than ID or IM injected Fluarix without Congo Red. Application of MPAs to the rump area of the animals is slightly superior to the application of MAPs to the rat car. According to feedback from rat biology, correct application to the car of the animals was difficult to achieve due to the large size of the MAPs compared to the small size of the rat car. These studies demonstrate that transdermal vaccinations against influenza viruses using the MicroArray Patches disclosed herein were as efficient as intradermal immunizations and standard intramuscular immunizations.

### Example 6—Adjuvants

Three different vaccine formulations containing purified Hemagglutinin-protein and three different adjuvants: Pam3CSK4, Gardiquimod, and beta-glucan peptide, are tested on MicroArrays for stability. In order to prepare the vaccine formulations, purified protein from eEnzyme® (Hemagglutinin (HA) (A/California/06/2009) (H1N1) (SWINE FLU 2009) Protein) is used as purchased at a concentration of 1 mg/ml. The protein solution is not concentrated further due to its low volume of only 100 μl. HA protein is mixed with Trehalose, and the three different adjuvants, with or without Trypan Blue and NP40. The dispensing of these formulations into reservoirs of microtips on MicroArrays using a microfluidic device is tested beforehand with BSA (bovine serum albumin) instead of HA. The three adjuvants being tested are: Pam3CSK4, Gardiquimod and Beta-glucan peptide. The formulations for each group are as follows: Formulation 1) 0.5 μg HA/7.5% Trehalose/1.25 μg Pam3CSK4/0.125% NP40/0.12% Trypan Blue; Formulation 2) 0.25 μg HA/7.5% Trehalose/0.6 μg Gardiquimod/0.12% Trypan Blue; and Formulation 3) 0.5 μg HA/7.5% Trehalose/2.5 μg Beta-glucan peptide/0.125% NP40.

HA protein (0.25 μg-0.5 μg) is dispensed (4×5 nL or 8×5 nL, i.e. 20 nL or 40 nL) into reservoirs of microtips in 25% polished 5×5−1 (large) MicroArrays, using a microfluidic dispenser. MicroArrays are stored at room temperature after the protein solution is deposited into the reservoirs of the microtips in the MicroArrays, sealed in slide mailers and foil bags with four H₂O-scavenger sachets per foil bag.

A first group of mice is administered Formulation 1 (i.e. the HA protein formulation with Pam3CSK4 as an adjuvant) via a MicroArray. A second group of mice is administered Formulation 2 (i.e. the HA protein formulation with Gardiquimod as an adjuvant) via a MicroArray. A third group of mice is administered Formulation 3 (i.e. the HA protein formulation with beta-glucan peptide as an adjuvant) via a MicroArray. Mouse sera is collected and analyzed post-immunization.

Total titer values after administration of the influenza vaccines are analyzed by enzyme-linked immunosorbent assays (ELISA). The ELISA tests are conducted as follows: A plate is coated overnight at 4° C. with HA protein at 0.5 μg/ml. The plate is washed three times with TBST (tris-buffered saline and Tween 20) and is blocked with 5% BSA in TBS (tris-buffered saline) for 1 hour at room temperature. After washing, mouse sera (1:100-1:12500) and a positive control (1:62,500-1:7,812,500; monoclonal anti-HA-antibody, ImmuneTech) in 1% BSA/TBST is added and incubated for 2 hours at room temperature, followed by washing. Anti-mouse-SA antibody, 1:5000 in 1% BSA/TBST, is added and incubated for 1 hour at room temperature. The plate is washed again and then incubated with anti-SA (1:200) in 1% BSA/TBST for 20 minutes at room temperature. After washing, substrate is added and incubated for 30 minutes at room temperature. The reaction is stopped by addition of 50 μl 2N sulfuric acid.

All three formulations dry within a few minutes of being dispensed into the reservoirs of the microtips on the MicroArrays, using an automated, low volume (i.e. nanoliter) microfluidic dispenser. Titer values in mouse sera from all three groups of mice indicate a strong immune response and thus, indicate that all three formulations remain stable and viable after drying.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-108. (canceled)

109. A MicroArray comprising:
a plurality of microtips formed in a substrate, wherein each microtip of the plurality of microtips comprises a reservoir and a hinge portion, the substrate having a first thickness and each microtip having a second thickness,
wherein the first thickness of the substrate remains substantially the same as the second thickness of the microtip except where a reservoir exists and where the hinged portion exists,
wherein each microtip has a proximal end and a distal end, such that the hinge portion is at the proximal end of the microtip where the microtip remains attached to the substrate,
wherein the hinged portion is thinner than the thickness of the starting substrate.

110. The MicroArray of claim 109, wherein the substrate is substantially planar.

111. The MicroArray of claim 109, wherein the hinged portion has a third thickness that is up to 80% less than the thickness of the substrate.

112. The MicroArray of claim 109, wherein each of the reservoirs on each of the microtips is loaded with a substance, wherein the substance is selected from the group consisting of an active pharmaceutical ingredient, a mixture of active pharmaceutical ingredients, and a vaccine.

113. The MicroArray of claim 112, wherein the substance comprises RNA.

114. The MicroArray of claim 112, wherein the substance is formulated as a sugar glass or sugar crystal.

115. The MicroArray of claim 114, wherein the substance is formulated as a sugar glass and wherein the sugar glass comprises sucralose, glucose, galactose, fructose, trehalose, maltose, or a combination thereof.

116. The MicroArray of claim 109, wherein the microtips comprise a microtip density of from 10 to 100 microtips per square centimeter of substrate surface area.

117. The MicroArray of claim 109, wherein the substrate comprises titanium, aluminum, stainless steel, nickel, iron, gold, copper, ruthenium, rhodium, palladium, silver, platinum, polyethylene, polypropylene, polyethylene terephthalate, polycarbonate, Teflon, or mixtures thereof.

118. The MicroArray of claim 109, wherein the substrate comprises a 25 micron to 150 micron thick metal sheet.

119. The MicroArray of claim 118, wherein the metal is chosen from the group consisting of titanium, stainless steel, nickel, and mixtures thereof.

120. The MicroArray of claim 109, wherein the substantially planar substrate comprises a plastic sheet of about 0.5 micron to 200 micron thickness.

121. The MicroArray of claim 120, wherein the plastic is a thermoplastic material.

122. The MicroArray of claim 109, wherein a microtip of the plurality of microtips comprises a beveled edge.

123. The MicroArray of claim 122, wherein the beveled edge is a double beveled edge, a top beveled edge, a bottom beveled edge, a double concave beveled edge, a top concave beveled edge, a bottom concave beveled edge, or a concave beveled edge.

124. The MicroArray of claim 109, wherein each of the microtips projects at an angle relative to the substrate.

125. The MicroArray of claim 124, wherein the angle is from about 50° to about 90° relative to the substrate.

126. The MicroArray of claim 109, wherein each of the microtips has a length of about 10 to about 1000 microns.

127. The MicroArray of claim 126, wherein each of the microtips has a length of about 600 to about 800 microns.

128. The MicroArray of claim 109, wherein the plurality of microtips is arranged in rows and columns on the substrate.
